(19) United States
(12) Patent Application Publication
(10) Pub. No.: US 2026/0184662 A1
(43) Pub. Date: Jul. 2, 2026

(54) COMPOSITE OF ACID AND SALT, AND PREPARATION METHOD THEREFOR

(71) Applicant: NANJING NUTRABUILDING BIO-TECH CO., LTD., Nanjing (CN)

(72) Inventors: Liang RONG, Nanjing (CN); Jinjian ZHU, Nanjing (CN); Youjian CHEN, Nanjing (CN); Long JIANG, Nanjing (CN); Xi ZHU, Nanjing (CN); Kylin LIAO, Nanjing (CN); Shawn WELLS, Nanjing (CN)

(21) Appl. No.: 19/115,649

(22) PCT Filed: Sep. 26, 2023
(86) PCT No.: PCT/CN2023/121701

§ 371 Date: Mar. 2, 2026

(30) Foreign Application Priority Data
Sep. 27, 2022 (WO) PCT/CN2022/121699

(51) Int. Cl. C07C 59/01; C07C 51/41; C07C 51/43; C07C 51/47

(52) U.S. Cl. CPC C07C 59/01; C07C 51/412; C07C 51/43; C07C 51/47; C07B 2200/13

(57) ABSTRACT

The invention discloses a complex of acid and salt and a preparation method thereof. The complex provided by the invention effectively solves the acid/salt load and gastrointestinal side effects associated with the existing products, has good taste comfort, and has a good ketogenic effect, and can be better used as a dietary or nutritional supplement.

### FIELD OF THE INVENTION

This invention belongs to the technical field of dietary or nutritional supplements, specifically relates to a complex of acid and salt and a preparation method thereof.

### BACKGROUND OF THE INVENTION

At present, people's demand for dietary or nutritional supplements is increasing, aiming at improving individual health and reducing disease risk. Athletes also use dietary or nutritional supplements to improve their strength and performance.

While normally human body would rely on glucose for energy, when the supply of glucose is too low for the body's energy needs, such as during periods of prolonged exercise, starvation, or absence of dietary carbohydrates, the body will turn to consume fat as fuel. Since the brain and central nervous system cannot directly use fat for energy, the liver produces ketone bodies (also known as ketones) from fatty acids as an alternative fuel source, which are then released into the blood/plasma. Ketones not only provide fuel for the brain, but are also used by the skeletal and heart muscle. The metabolism of ketone bodies is associated with several beneficial effects, including anticonvulsant effects, enhanced brain metabolism, neuroprotection, muscle sparing properties, and improved cognitive and physical performance. Science-based improvements in efficiency of cellular metabolism, managed through ketone supplementation, can have beneficial impacts on physical, cognitive health, and psychological health, and a long-term impact on health with respect to common avoidable diseases such as obesity, cardiovascular disease, neurodegenerative diseases, diabetes, and cancer.

Despite the many health advantages of pursuing a ketogenic diet or lifestyle and maintaining a state of nutritional ketosis, there remain significant barriers to pursuing and maintaining a ketogenic state. One of these barriers is the difficulty of transitioning into a ketogenic state. The fastest endogenous way to entering ketosis through depleting glucose stores in the body is by fasting combined with exercise. This is physically and emotionally demanding and is extremely challenging even for the most motivated and disciplined.

A large number of studies on exogenous ketones show that the intake of compounds that can increase the level of ketone bodies in blood can bring various clinical benefits, including enhancing physical and cognitive abilities and treating cardiovascular diseases, diabetes, neurodegenerative diseases and epilepsy. Therefore, it is desirable to provide ketone bodies as energy directly to humans or animals. Dietary or nutritional supplements may contain carboxylic acids, such as β-hydroxybutyric acid (also known as 3-hydroxybutyric acid or BHB), which is one of the three main ketone bodies (namely acetoacetate, acetone and BHB).

However, currently known ingestible exogenous ketone bodies have the disadvantage of limiting their use. β-hydroxybutyric acid is a source of exogenous ketones, and its well-known problem is extremely strong acidity. Because of this acidity, the amount and concentration of β-hydroxybutyric acid used in ingestible form are limited. By converting β-hydroxybutyric acid into its sodium, magnesium, calcium and potassium salts, the acidity problem of D-BHB acid has been solved in some applications. However, although salt can solve the problem of acidity, it is easy to lead to electrolyte imbalance due to salt overload, and the use of ketone salt is also limited to a very small amount, which is not only a small dose, but also unpleasant. When carboxylic acid and salt are simply physically mixed, the above problems still exist, and it is difficult to achieve uniform mixing.

Therefore, in order to solve the problems of strong acidity and high hygroscopicity of the existing acid, high salt load, gastrointestinal side effects and unpleasant taste of the existing salt, it is necessary to further find substances that can effectively avoid or balance the above problems, so as to be better used as ketogenic substances in diet or nutritional supplements, especially for the preparation of solid particles and the application of particles.

### SUMMARY OF THE INVENTION

In one aspect, the present invention provides a complex comprising 3-hydroxybutyric acid; and one or more of potassium 3-hydroxybutyrate, calcium 3-hydroxybutyrate, or magnesium 3-hydroxybutyrate.

In some embodiments, anions in the complex structure include 3-hydroxybutyric acid anions; and cations include one or more of potassium ions, calcium ions and magnesium ions, as well as hydrogen ions.

In some embodiments, the ratio of 3-hydroxybutyric acid to one or more of potassium 3-hydroxybutyrate, calcium 3-hydroxybutyrate or magnesium 3-hydroxybutyrate is 1:10 to 10:1.

In some embodiments, the complex contains not less than 50% of R configuration, not more than 50% of S configuration; or more than 50% of S configuration, less than 50% of R configuration.

In some embodiments, the complex is 3-hydroxybutyric acid·potassium 3-hydroxybutyrate or 3-hydroxybutyric acid·calcium 3-hydroxybutyrate or 3-hydroxybutyric acid·magnesium 3-hydroxybutyrate or mixture thereof.

In some embodiments, the complex has the following structure:

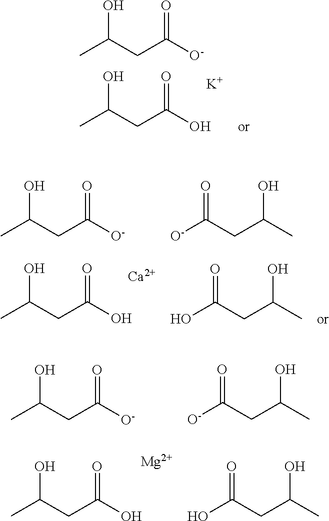

or mixture thereof.

In one aspect, the present invention provides a 3-hydroxybutyric acid·3-hydroxybutyrate salt complex, and the salt is a potassium salt, a calcium salt and/or a magnesium salt.

In some embodiments, the complex is a R-3-hydroxybutyric acid·R-3-hydroxybutyrate salt complex and/or a S-3-hydroxybutyric acid·S-3-hydroxybutyrate salt complex.

In some embodiments, the complex contains not less than 50% of R-3-hydroxybutyric acid·R-3-hydroxybutyrate salt, not more than 50% of S-3-hydroxybutyric acid·S-3-hydroxybutyrate salt; or more than 50% of S-3-hydroxybutyric acid·S-3-hydroxybutyrate salt, less than 50% of R-3-hydroxybutyric acid·R-3-hydroxybutyrate salt.

In some embodiments, the complex has the following structure:

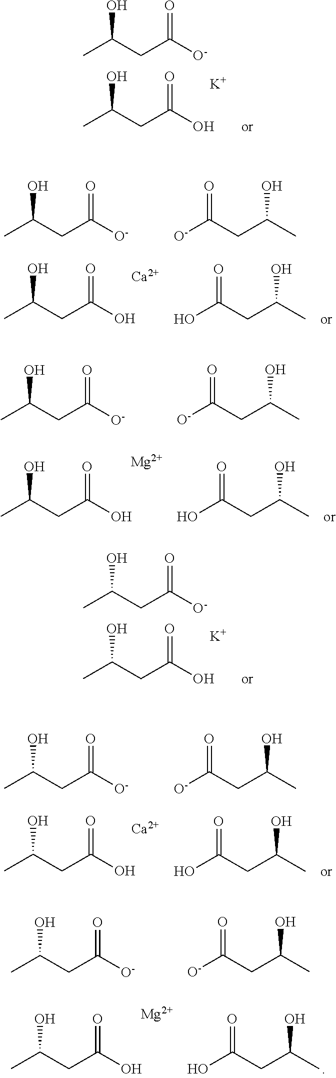

In some embodiments, the complex is in crystalline form.

In some embodiments, the X-ray powder diffraction pattern of the potassium salt complex comprises peaks at diffraction angles (2θ) of 6.7±0.2°, 19.6±0.2°, 24.9±0.2°, and 27.1±0.2°.

In some embodiments, the X-ray powder diffraction pattern of the potassium salt complex further comprises one or more peaks at diffraction angles (2θ) of 13.4±0.2°, 21.4±0.2°, 26.0±0.2°, 32.5±0.2°.

In some embodiments, the X-ray powder diffraction pattern of the potassium salt complex further comprises one or more peaks at diffraction angles (2θ) of 20.2±0.2°, 23.4±0.2°, 28.2±0.2°, 34.0±0.2°.

In some embodiments, the X-ray powder diffraction pattern of the potassium salt complex is shown in FIG. 1.

In some embodiments, the infrared spectrum of the potassium salt complex has the following absorption bands, expressed in reciprocal wavelengths (cm⁻¹) (±2 cm⁻¹): 2972, 2933, 1715, 1574, 1304, 1196, 1126, 1065, 951, 854, 474.

In some embodiments, the infrared spectrum of the calcium salt complex has the following absorption bands, expressed in reciprocal wavelengths (cm⁻¹) (±2 cm⁻¹): 2974, 2936, 1715, 1558, 1506, 1300, 1196, 1126, 1065, 951, 854, 422.

In some embodiments, the infrared spectrum of the magnesium salt complex has the following absorption bands, expressed in reciprocal wavelengths (cm⁻¹) (±2 cm 1): 2976, 2936, 1713, 1321, 1207, 1088, 957, 912, 826, 625, 554, 411.

In some embodiments, the complex is prepared as food, beverage, supplement or pharmaceutical preparation.

In another aspect, the present invention provides a 3-hydroxybutyric acid·3-hydroxybutyrate salt complex, which is obtained by the following method:
(1) a substance B is obtained by one of the following methods: mixing 3-hydroxybutyric acid and 3-hydroxybutyrate salt; or adding 3-hydroxybutyric acid into an aqueous solution of an alkaline compound, stirring, removing water, and evaporating to near dryness; or reacting alkyl 3-hydroxybutyrate with water under heating in the presence of a catalyst, cooling and filtering, adding an aqueous solution of an alkaline compound to the filtrate, distilling under reduced pressure to remove water, and evaporating to near dryness;
(2) adding one or more solvents selected from the following into the substance B obtained in step (1): water, THF, DMF, DMSO, DMAC, alcohol, halogenated hydrocarbon, ketone, ester, stirring and cooling, to precipitate a solid;
(3) filtering out the solid and drying, to obtain the complex.

In some embodiments, the complex is a R-3-hydroxybutyric acid·R-3-hydroxybutyrate salt complex and/or a S-3-hydroxybutyric acid·S-3-hydroxybutyrate salt complex.

In some embodiments, the complex is in crystalline form.

In another aspect, the present invention provides a method for preparing the above-mentioned complex, including the following steps:
(1) a substance B is obtained by one of the following methods: mixing 3-hydroxybutyric acid and 3-hydroxybutyrate salt; or adding 3-hydroxybutyric acid into an aqueous solution of an alkaline compound, stirring, removing water, and evaporating to near dryness; or reacting alkyl 3-hydroxybutyrate with water under heating in the presence of a catalyst, cooling and filtering, adding an aqueous solution of an alkaline compound to the filtrate, distilling under reduced pressure to remove water, and evaporating to near dryness;
(2) adding one or more solvents selected from the following into the substance B obtained in step (1): water, THF, DMF, DMSO, DMAC, alcohol, halogenated hydrocarbon, ketone, ester, stirring and cooling, to precipitate a solid;
(3) filtering out the solid and drying, to obtain the complex.

In some embodiments, in step (2), the alcohol is methanol, ethanol, isopropanol, n-butanol; the halogenated hydrocarbon is chlorobenzene, dichlorobenzene, dichloromethane; the ketone is acetone, methyl butanone, methyl isobutyl ketone; the ester is ethyl acetate, isopropyl acetate, n-butyl acetate, isobutyl acetate.

In some embodiments, in step (1), the alkaline compound is hydroxide, carbonate, bicarbonate, methanol, acetate or formate of potassium, calcium or magnesium; the alkyl 3-hydroxybutyrate is methyl 3-hydroxybutyrate, ethyl 3-hydroxybutyrate, propyl 3-hydroxybutyrate, isopropyl 3-hydroxybutyrate, butyl 3-hydroxybutyrate, isobutyl 3-hydroxybutyrate.

In another aspect, the present invention provides a composition comprising an effective amount of the above-mentioned complex, and a pharmaceutically acceptable carrier.

In some embodiments, the composition is used as a ketogenic substance.

In some embodiments, the composition is prepared as food, beverage, supplement or pharmaceutical preparation.

In another aspect, the present invention provides use of the complex, the complex is used for preparing a ketogenic substance for increasing or maintaining blood ketone level of a subject.

In some embodiments, the ketogenic substance can be used as nutritional supplement, energy therapy, medical treatment or strength and/or endurance exercise supplement.

In another aspect, the present invention provides use of a composition in preparing a ketogenic substance for increasing or maintaining blood ketone level of a subject, the composition comprises the complex of the invention, and a pharmaceutically acceptable carrier.

In some embodiments, the ketogenic substance can be used as nutritional supplement, energy therapy, medical treatment or strength and/or endurance exercise supplement.

In another aspect, the present invention provides a complex of acid and salt, wherein the acid comprises propionic acid, butyric acid, pentanoic acid, hexanoic acid and hydroxycarboxylic acid; and the salt comprises sodium salt, potassium salt, calcium salt and/or magnesium salt.

In some embodiments, the hydroxycarboxylic acid is 3-hydroxypentanoic acid (BHP).

In some embodiments, anions in the complex structure include 3-hydroxypentanoic acid anions; and cations include sodium ions and hydrogen ions.

In some embodiments, the ratio of 3-hydroxypentanoic acid to sodium 3-hydroxypentanoate is 1:10 to 10:1.

In some embodiments, the complex contains not less than 50% of R configuration, not more than 50% of S configuration; or more than 50% of S configuration, less than 50% of R configuration.

In some embodiments, the complex has the following structure:

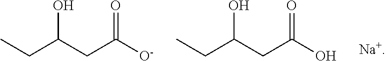

In some embodiments, the complex is in crystalline form.

In some embodiments, the complex is a R-3-hydroxypentanoic acid·sodium R-3-hydroxypentanoate and/or a S-3-hydroxypentanoic acid sodium S-3-hydroxypentanoate.

In some embodiments, the complex contains not less than 50% of R-3-hydroxypentanoic acid·sodium R-3-hydroxypentanoate, not more than 50% of S-3-hydroxypentanoic acid·sodium S-3-hydroxypentanoate; or more than 50% of S-3-hydroxypentanoic acid·sodium S-3-hydroxypentanoate, less than 50% of R-3-hydroxypentanoic acid·sodium R-3-hydroxypentanoate.

In some embodiments, the complex has the following structure:

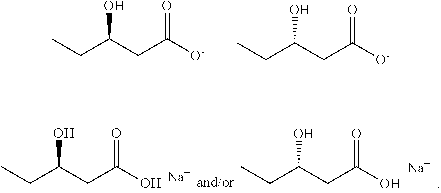

In some embodiments, the infrared spectrum of the complex has the following absorption bands, expressed in reciprocal wavelengths (cm⁻¹) (±2 cm⁻¹): 2968, 2880, 1715, 1558, 1404, 1065, 982, 912, 874, 783, 473, 426.

In some embodiments, the complex is prepared as food, beverage, supplement or pharmaceutical preparation.

In another aspect, the present invention provides a complex of acid and salt, which is obtained by the following method:
(1) a substance C is obtained by one of the following methods: mixing an acid and a corresponding salt; or adding an acid into an aqueous solution of an alkaline compound, stirring, removing water, and evaporating to near dryness; or reacting an alkyl ester of acid with water under heating in the presence of a catalyst, cooling and filtering, adding an aqueous solution of an alkaline compound to the filtrate, distilling under reduced pressure to remove water, and evaporating to near dryness;
(2) adding one or more solvents selected from the following into the substance C obtained in step (1): water, THF, DMF, DMSO, DMAC, alcohol, halogenated hydrocarbon, ketone, ester, stirring and cooling, to precipitate a solid;
(3) filtering out the solid and drying, to obtain the complex.

In some embodiments, the complex is propionic acid·sodium propionate, propionic acid·potassium propionate, propionic acid·calcium propionate, propionic acid·magnesium propionate, butyric acid·sodium butyrate, butyric acid·potassium butyrate, butyric acid·calcium butyrate, butyric acid·magnesium butyrate, pentanoic acid·sodium pentanoate, pentanoic acid·potassium pentanoate, pentanoic acid·calcium pentanoate, pentanoic acid·magnesium pentanoate, hexanoic acid·sodium hexanoate, hexanoic acid·potassium hexanoate, hexanoic acid·calcium hexanoate, hexanoic acid·magnesium hexanoate, 3-hydroxypentanoic acid·sodium 3-hydroxypentanoate, 3-hydroxypentanoic acid·potassium 3-hydroxypentanoate, 3-hydroxypentanoic acid·calcium 3-hydroxypentanoate, 3-hydroxypentanoic acid·magnesium 3-hydroxypentanoate.

In some embodiments, the complex is in crystalline form.

In another aspect, the present invention provides a method for preparing the above-mentioned complex, including the following steps:
(1) a substance C is obtained by one of the following methods: mixing an acid and a corresponding salt; or adding an acid into an aqueous solution of an alkaline compound, stirring, removing water, and evaporating to near dryness; or reacting an alkyl ester of acid with water under heating in the presence of a catalyst, cooling and filtering, adding an aqueous solution of an alkaline compound to the filtrate, distilling under reduced pressure to remove water, and evaporating to near dryness;
(2) adding one or more solvents selected from the following into the substance C obtained in step (1): water, THF, DMF, DMSO, DMAC, alcohol, halogenated hydrocarbon, ketone, ester, stirring and cooling, to precipitate a solid;
(3) filtering out the solid and drying, to obtain the complex.

In some embodiments, in step (2), the alcohol is methanol, ethanol, isopropanol, n-butanol; the halogenated hydrocarbon is chlorobenzene, dichlorobenzene, dichloromethane; the ketone is acetone, methyl butanone, methyl isobutyl ketone; the ester is ethyl acetate, isopropyl acetate, n-butyl acetate, isobutyl acetate.

In some embodiments, in step (1), the alkaline compound is hydroxide, carbonate, bicarbonate, methanol, acetate or formate of sodium, potassium, calcium or magnesium; the alkyl ester of acid is methyl ester, ethyl ester, propyl ester, isopropyl ester, butyl ester and isobutyl ester of acid.

In another aspect, the present invention provides a composition comprising an effective amount of the above-mentioned complex, and a pharmaceutically acceptable carrier.

In some embodiments, the composition is used as a ketogenic substance.

In some embodiments, the composition is prepared as food, beverage, supplement or pharmaceutical preparation.

In another aspect, the present invention provides use of the above complex, the complex is used for preparing a ketogenic substance for increasing or maintaining blood ketone level of a subject.

In some embodiments, the ketogenic substance can be used as nutritional supplement, energy therapy, medical treatment or strength and/or endurance exercise supplement.

In another aspect, the present invention provides use of a composition in preparing a ketogenic substance for increasing or maintaining blood ketone level of a subject, the composition comprises the above complex, and a pharmaceutically acceptable carrier.

In some embodiments, the ketogenic substance can be used as nutritional supplement, energy therapy, medical treatment or strength and/or endurance exercise supplement.

Compared with the prior art, the complex of the invention has the beneficial effects that the complex of the invention has no bad smell and effectively avoids the problems of acidity, hygroscopicity, salt load, intestinal side effects, electrolyte imbalance and the like; and compared with other exogenous ketones, the complex has excellent adaptability. When administered to a subject, the complex shows a better comprehensive effect than acid or salt alone or simply physically mixed components. The complex of the invention solves the problems of strong acidity, intestinal side effects and high hygroscopicity of acid; and at the same time solves the problem of electrolyte imbalance caused by high salt load of salt, and there is no problem of uneven physical mixing, so it can be widely used as a ketogenic substance in dietary supplements or food fields.

### BRIEF DESCRIPTION OF THE DRAWINGS

FIG. 1 shows an XRPD pattern of the 3-hydroxybutyric acid·potassium 3-hydroxybutyrate complex (Complex I) of the present invention.

FIG. 2A shows an infrared spectrogram (IR) of the 3-hydroxybutyric acid·potassium 3-hydroxybutyrate complex (Complex I) of the present invention.

FIG. 2B shows an infrared spectrogram (IR) of the 3-hydroxybutyric acid·calcium 3-hydroxybutyrate complex (Complex II) of the present invention.

FIG. 2C shows an infrared spectrogram (IR) of the 3-hydroxybutyric acid·magnesium 3-hydroxybutyrate complex (Complex III) of the present invention.

FIG. 2D shows an infrared spectrogram (IR) of the 3-hydroxypentanoic acid·sodium 3-hydroxypentanoate complex (Complex IV) of the present invention.

FIG. 3 shows a Raman spectrogram of the 3-hydroxybutyric acid·potassium 3-hydroxybutyrate complex (Complex I) of the present invention.

FIG. 4A shows a TGA diagram of the 3-hydroxybutyric acid·potassium 3-hydroxybutyrate complex (Complex I) of the present invention.

FIG. 4B-1 to 4B-3 show TGA diagrams of the 3-hydroxybutyric acid·calcium 3-hydroxybutyrate complex (Complex II) of the present invention.

FIG. 4C-1 to 4C-3 show TGA diagrams of the 3-hydroxybutyric acid·magnesium 3-hydroxybutyrate complex (Complex III) of the present invention.

FIG. 4D shows a TGA diagram of the 3-hydroxypentanoic acid·sodium 3-hydroxypentanoate complex (Complex IV) of the present invention.

FIG. 5 shows a DSC thermogram of the 3-hydroxybutyric acid·potassium 3-hydroxybutyrate complex (Complex I) of the present invention.

### DETAILED DESCRIPTION OF THE INVENTION

Reference will now be made in detail to the preferred embodiments of the invention, examples of which are further illustrated. While the invention will be described in conjunction with the preferred embodiments, it will be understood that they are not intended to limit the invention to these embodiments. On the contrary, the invention is intended to cover alternatives, modifications, and equivalents, which may be included within the spirit and scope of the invention as defined by the claims. Furthermore, in the detailed description of the present invention, numerous specific details are set forth in order to provide a thorough understanding of the present invention. However, it will be obvious to one of ordinary skill in the art that the present invention may be practiced without these specific details. In other instances, well known methods, procedures, components, and other features have not been described in detail as not to unnecessarily obscure aspects of the present invention.

The 3-hydroxybutyric acid·3-hydroxybutyrate salt complex of the invention comprises 3-hydroxybutyric acid and 3-hydroxybutyrate salt in any suitable ratio, and the complex can be hydrate with fixed water ratio, non-hydrate, and corresponding crystal forms. The acid·salt complex of the invention comprises 3-hydroxypentanoic acid (BHP), propionic acid, butyric acid, pentanoic acid and/or hexanoic acid and sodium salt, potassium salt, calcium salt and/or magnesium salt in any suitable ratio, and the complex can be hydrate with fixed water ratio, non-hydrate, and corresponding crystal forms.

As used herein, the term “or” is meant to include both “and” and “or.” In other words, the term “or” may also be replaced with “and/or.”

As used herein, the singular forms “a,” “an” and “the” are intended to include the plural forms as well, unless the context clearly indicates otherwise.

As used herein, the term “comprise” or “include” and their conjugations, refer to a situation wherein said terms are used in their non-limiting sense to mean that items following the word are included, but items not specifically mentioned are not excluded. It also encompasses the more limiting verb ‘to consist essentially of’ and ‘to consist of’.

As use herein, the terms “about” and “approximately” provide numerical flexibility by providing endpoints where a given value can be “slightly above” or “less than”. The flexibility of this term can be determined by specific variables and based on experience and related descriptions herein within the knowledge of those skilled in the art.

β-hydroxybutyrate, also known as 3-hydroxybutyrate, BHB, or BHB, refers to a compound with the general formula CH₃CH₂OHCH₂COOH. “Beta-hydroxybutyrate derivative” represents a compound having chemical structure shown below, where X can be hydrogen, metal ion, amino cation (e.g., amino acid), and the like:

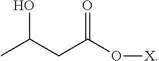

When X is a hydrogen, the compound is beta-hydroxybutyric acid. When X is a metal ion or an amino cation, the compound is a beta-hydroxybutyrate salt. The foregoing compounds can be in any desired physical form, such as crystalline, powder, solid, liquid, solution, suspension, or gel.

As used herein, the term “administration” refers to the process of delivering a disclosed composition or active ingredient to a subject. The complexes of the invention can be administered in a variety of suitable ways, including orally, intragastrically, and parenterally (e.g., intravenous and intraarterial as well as other suitable parenteral routes), and the like, so as to exert the desired effects. The complex of the invention may be administered to a subject in an effective dosage and/or in frequencies to induce or sustain ketosis. In some embodiments, a single dose will include an amount of about 1-50 grams, or about 2-40 grams, or about 5-30 grams, or about 10-20 grams, about 0.5-25 grams, or about 0.75-20 grams, or about 1-15 grams, or about 1.5-12 grams. In some embodiments, multiple doses of complex are administered over a period of time. The frequency of administration of the complex can vary depending on any of a variety of factors, such as timing of treatment from previous treatments, objectives of the treatment, and the like. The duration of administration of the complex (e.g., the period of time over which the agent is administered), can vary depending on any of a variety of factors, including subject response, desired effect of treatment, etc.

As used herein, the term “effective amount” refers to the amount required to achieve an effect as taught herein. The amount to be administered can vary according to factors such as the degree of susceptibility of the individual, the age, sex, and weight of the individual, idiosyncratic responses of the individual, and the like. In accordance with the present disclosure, a suitable single dose size is a dose that is capable of achieve the above effects when administered one or more times over a suitable time period.

As used herein, the term “pharmaceutically acceptable” means pharmaceutically, physiologically, alimentarily, and/or nutritionally acceptable, and refers to those compositions or combinations of agents, materials, or compositions, and/or their dosage forms, which are within the scope of sound medical judgment, suitable for use in contact with the tissues of human beings and animals without excessive toxicity, irritation, allergic response, or other problem or complication, commensurate with a reasonable benefit/risk ratio.

In some embodiments, because beta-hydroxybutyrate may be R-beta-hydroxybutyrate enantiomer, which is endogenously produced by a mammal during ketosis, administering the R-beta-hydroxybutyrate enantiomer to a subject provides an additional quantity and/or increased blood plasma level that can be immediately utilized by the body, such as for producing energy (e.g., as an alternative energy source to glucose).

The complexes and/or compositions of the present invention may be used for preparing a ketogenic substance for increasing or maintaining blood ketone level of a subject, and increasing ketone body level in a subject, including inducing and/or sustaining a state of elevated ketone bodies at a desired level, such as ketosis, in a subject to which it is administered. “Ketosis” refers to a subject having blood ketone levels within the range of about 0.5 mmol/L and about 16 mmol/L in a subject. Ketosis may improve mitochondrial function, decrease reactive oxygen species production, reduce inflammation and increase the activity of neurotrophic factors. “Keto-adaptation” refers to prolonged nutritional ketosis (>1 week) to achieve a sustained nonpathological “mild ketosis” or “therapeutic ketosis.” In some cases, “elevated ketone body level” may not mean that a subject is in a state of “clinical ketosis” but nevertheless has an elevated supply of ketones for producing energy and/or for carrying out other beneficial effects of ketone bodies.

The administration of the complexes and/or compositions of the present invention can increase or maintain blood ketone level of a subject, and as a ketogenic substance, produce one or more desired effects, including but not limited to appetite suppression, weight loss, fat loss, reduced blood glucose level, improved mental alertness, increased physical energy, improved cognitive function, reduction in traumatic brain injury, reduction in effect of diabetes, improvement of neurological disorder, reduction of cancer, reduction of inflammation, anti-aging, antiglycation, reduction in epileptic seizer, improved mood, increased strength, increased muscle mass, or improved body composition.

In some embodiments, the complex of the present invention can be prepared into a composition together with an alimentarily or pharmaceutically acceptable carrier. In the present invention, the application form of providing composition involves liquid or solid fillers, diluents, excipients, solvents or encapsulate materials. Each carrier must be “acceptable”, which means that it is compatible with other components of the composition and harmless to the subject, that is, suitable for consumption or nutritionally acceptable. The carriers include non-toxic and compatible substances commonly used in health foods, dietary supplements and pharmaceutical preparations, such as sugar, starch, cellulose and its derivatives, powdered tragacanth, malt, gelatin, talc, oil, diol, polyol, ester, agar, alginic acid, pyrogen free water, isotonic saline, etc.

In some embodiments, the complexes of the present invention can be administered together with other supplements, such as vitamins, minerals, nootropics, and others known in the art. Examples of vitamins, minerals and herbal supplements that can be added to the ketogenic compositions include one or more of vitamin A, vitamin C, vitamin D3, vitamin E, niacin, vitamin B6, folic acid, 5-MTHF, vitamin B12, iodine, zinc, copper, manganese, chromium, caffeine, theobromine, theacrine, methylliberine, huperzine A, epicatechins, and enzymes

In some embodiments, the complexes of the present invention may be provided as a solid or powder form. The compositions in such solid form may be formulated so as to provide for sufficient ease of handling and manufacturability. The complexes may be provided as a liquid, such as in the form of a shot or mouth spray for fast delivery and absorption. Liquid forms may include one or more liquid carriers, such as water, ethanol, glycerin, propylene glycol, 1,3-propandiol, and the like.

In some embodiments, the complex of the present invention can be used as suppository, tablet, pill, granule, powder, film, capsule, beverage, aerosol, alcohol, tincture, tonic, liquid suspension or syrup.

The complex and/or composition of the present invention can be prepared as food and beverage products for human consumption, as well as nutritional supplements, energy therapy, medical treatment or strength and/or endurance exercise supplements as ketogenic substances, thereby providing a dietary source of exogenous ketones and raising or maintaining blood ketone level of a subject. The obtained product can show balance effects of reduced acidity, lower hygroscopicity, better taste, better palatability, uniform appearance and good ketogenic effect, and has no problems of intestinal side effects, electrolyte imbalance and high salt load.

The following examples are illustrative of select embodiments of the present invention and are not meant to limit the scope of the invention.

### Preparation of Complexes of the Present Invention

### Example 1. Preparation of R-3-hydroxybutyric acid·potassium R-3-hydroxybutyrate Complex (Complex I)

110 g of R-3-hydroxybutyric acid, 110 g of potassium R-3-hydroxybutyrate, 440 mL dichloromethane were added into a 1-L reaction bottle, heated to 40° C. It was stirred and dissolved, cooled to 0-10° C.; the solid was precipitated, filtered, and dried at 40° C., to obtain 160 g of R-3-hydroxybutyric acid·potassium R-3-hydroxybutyrate complex.

### Example 2. Preparation of R-3-hydroxybutyric acid·potassium R-3-hydroxybutyrate Complex (Complex I)

104 g of R-3-hydroxybutyric acid, 142 g of potassium R-3-hydroxybutyrate, 440 mL acetone were added into a 1-L reaction bottle, heated to 60° C. It was stirred and dissolved, cooled to 0-10° C.; the solid was precipitated, filtered, and dried at 55° C., to obtain 200 g of R-3-hydroxybutyric acid·potassium R-3-hydroxybutyrate complex.

### Example 3. Preparation of R-3-hydroxybutyric acid·potassium R-3-hydroxybutyrate Complex (Complex I)

200 mL water and 28 g potassium hydroxide were added into a 1-L reaction bottle. It was stirred and dissolved, cooled to below 25° C., and then 104 g of R-3-hydroxybutyric acid was added. It was stirred for half an hour, and distilled under reduced pressure to remove water. After evaporation to near dryness, 200 mL of dichloromethane was added. It was stirred, cooled to 0-10° C.; the solid was precipitated, filtered, and dried at 40° C., to obtain 80 g of R-3-hydroxybutyric acid·potassium R-3-hydroxybutyrate complex.

### Example 4. Preparation of R-3-hydroxybutyric acid·potassium R-3-hydroxybutyrate Complex (Complex I)

200 mL water and 28 g potassium hydroxide were added into a 1-L reaction bottle. It was stirred and dissolved, cooled to below 25° C., and then 104 g of R-3-hydroxybutyric acid was added. It was stirred for half an hour, and distilled under reduced pressure to remove water. After evaporation to near dryness, 200 mL of acetone was added. It was stirred, cooled to 0-10° C.; the solid was precipitated, filtered, and dried at 55° C., to obtain 100 g of R-3-hydroxybutyric acid·potassium R-3-hydroxybutyrate complex.

### Example 5. Preparation of R-3-hydroxybutyric acid·potassium R-3-hydroxybutyrate Complex (Complex I)

60 g of methyl R-3-hydroxybutyrate, 360 mL water and 24 g of catalyst were added into a 1-L reaction bottle, heated at 90-95° C. for 24 hours until the reaction was complete. It was cooled to room temperature, and the catalyst was filtered off. 50 mL aqueous solution of 13 g of potassium hydroxide was added to the filtrate, and the water was distilled off under reduced pressure. After evaporation to near dryness, 100 mL of dichloromethane was added. It was stirred, cooled to 0-10° C.; the solid was precipitated, filtered, and dried at 40° C., to obtain 45 g of R-3-hydroxybutyric acid·potassium R-3-hydroxybutyrate complex.

### Example 6. Preparation of R-3-hydroxybutyric acid·potassium R-3-hydroxybutyrate Complex (Complex I)

60 g of methyl R-3-hydroxybutyrate, 360 mL water and 24 g of catalyst were added into a 1-L reaction bottle, heated at 90-95° C. for 24 hours until the reaction was complete. It was cooled to room temperature, and the catalyst was filtered off. 50 mL aqueous solution of 13 g of potassium hydroxide was added to the filtrate, and the water was distilled off under reduced pressure. After evaporation to near dryness, 100 mL of acetone was added. It was stirred, cooled to 0-10° C.; the solid was precipitated, filtered, and dried at 55° C., to obtain 52 g of R-3-hydroxybutyric acid·potassium R-3-hydroxybutyrate complex.

### Example 7. Preparation of 3-hydroxybutyric acid·potassium 3-hydroxybutyrate Complex (Complex I)

110 g of 3-hydroxybutyric acid, 110 g of potassium 3-hydroxybutyrate, 440 mL dichloromethane were added into a 1-L reaction bottle, heated to 40° C. It was stirred and dissolved, cooled to 0-10° C.; the solid was precipitated, filtered, and dried at 40° C., to obtain 152 g of 3-hydroxybutyric acid·potassium 3-hydroxybutyrate complex.

### Example 8. Preparation of 3-hydroxybutyric acid·potassium 3-hydroxybutyrate Complex (Complex I)

104 g of 3-hydroxybutyric acid, 142 g of potassium 3-hydroxybutyrate, 440 mL acetone were added into a 1-L reaction bottle, heated to 60° C. It was stirred and dissolved, cooled to 0-10° C.; the solid was precipitated, filtered, and dried at 55° C., to obtain 170 g of 3-hydroxybutyric acid·potassium 3-hydroxybutyrate complex.

### Example 9. Preparation of 3-hydroxybutyric acid·potassium 3-hydroxybutyrate Complex (Complex I)

200 mL water and 28 g of potassium hydroxide were added into a 1-L reaction bottle. It was stirred and dissolved, cooled to below 25° C., and then 104 g of 3-hydroxybutyric acid was added. It was stirred for half an hour, and distilled under reduced pressure to remove water. After evaporation to near dryness, 200 mL of acetone was added. It was stirred, cooled to 0-10° C.; the solid was precipitated, filtered, and dried at 55° C., to obtain 98 g of 3-hydroxybutyric acid·potassium 3-hydroxybutyrate complex.

### Example 10. Preparation of 3-hydroxybutyric acid·potassium 3-hydroxybutyrate Complex (Complex I)

200 mL water and 28 g of potassium hydroxide were added into a 1-L reaction bottle. It was stirred and dissolved, cooled to below 25° C., and then 104 g of 3-hydroxybutyric acid was added. It was stirred for half an hour, and distilled under reduced pressure to remove water. After evaporation to near dryness, 200 mL of dichloromethane was added. It was stirred, cooled to 0-10° C.; the solid was precipitated, filtered, and dried at 40° C., to obtain 82 g of 3-hydroxybutyric acid·potassium 3-hydroxybutyrate complex.

### Example 11. Preparation of 3-hydroxybutyric acid·potassium 3-hydroxybutyrate Complex (Complex I)

60 g of methyl 3-hydroxybutyrate, 360 mL water and 24 g of catalyst were added into a 1-L reaction bottle, heated at 90-100° C. for 24 hours until the reaction was complete. It was cooled to room temperature, and the catalyst was filtered off. 50 mL aqueous solution of 13 g of potassium hydroxide was added to the filtrate, and the water was distilled off under reduced pressure. After evaporation to near dryness, 100 mL of dichloromethane was added. It was stirred, cooled to 0-10° C.; the solid was precipitated, filtered, and dried at 40° C., to obtain 48 g of 3-hydroxybutyric acid·potassium 3-hydroxybutyrate complex.

### Example 12. Preparation of 3-hydroxybutyric acid·potassium 3-hydroxybutyrate Complex (Complex I)

60 g of methyl 3-hydroxybutyrate, 360 mL water and 24 g of catalyst were added into a 1-L reaction bottle, heated at 90-95° C. for 24 hours until the reaction was complete. It was cooled to room temperature, and the catalyst was filtered off. 50 mL aqueous solution of 13 g of potassium hydroxide was added to the filtrate, and the water was distilled off under reduced pressure. After evaporation to near dryness, 100 mL of acetone was added. It was stirred, cooled to 0-10° C.; the solid was precipitated, filtered, and dried at 55° C., to obtain 58 g of 3-hydroxybutyric acid·potassium 3-hydroxybutyrate complex.

### Example 13. Preparation of S-3-hydroxybutyric acid·potassium S-3-hydroxybutyrate Complex (Complex I)

110 g of R-3-hydroxybutyric acid, 110 g of potassium R-3-hydroxybutyrate, 440 mL dichloromethane were added into a 1-L reaction bottle, heated to 40° C. It was stirred and dissolved, cooled to 0-10° C.; the solid was precipitated, filtered, and dried at 40° C., to obtain 162 g of R-3-hydroxybutyric acid·potassium R-3-hydroxybutyrate complex.

### Example 14. Preparation of S-3-hydroxybutyric acid·potassium S-3-hydroxybutyrate Complex (Complex I)

104 g of S-3-hydroxybutyric acid, 142 g of potassium S-3-hydroxybutyrate, 440 mL acetone were added into a 1-L reaction bottle, heated to 60° C. It was stirred and dissolved, cooled to 0-10° C.; the solid was precipitated, filtered, and dried at 55° C., to obtain 186 g of S-3-hydroxybutyric acid·potassium S-3-hydroxybutyrate complex.

### Example 15. Preparation of S-3-hydroxybutyric acid·potassium S-3-hydroxybutyrate Complex (Complex I)

200 mL water and 28 g of potassium hydroxide were added into a 1-L reaction bottle. It was stirred and dissolved, cooled to below 25° C., and then 104 g of S-3-hydroxybutyric acid was added. It was stirred for half an hour, and distilled under reduced pressure to remove water. After evaporation to near dryness, 200 ml of dichloromethane was added. It was stirred, cooled to 0-10° C.; the solid was precipitated, filtered, and dried at 40° C., to obtain 78 g of S-3-hydroxybutyric acid·potassium S-3-hydroxybutyrate complex.

### Example 16. Preparation of S-3-hydroxybutyric acid·potassium S-3-hydroxybutyrate Complex (Complex I)

200 mL water and 28 g potassium hydroxide were added into a 1-L reaction bottle. It was stirred and dissolved, cooled to below 25° C., and then 104 g of S-3-hydroxybutyric acid was added. It was stirred for half an hour, and distilled under reduced pressure to remove water. After evaporation to near dryness, 200 mL of acetone was added. It was stirred, cooled to 0-10° C.; the solid was precipitated, filtered, and dried at 55° C., to obtain 90 g of S-3-hydroxybutyric acid·potassium S-3-hydroxybutyrate complex.

### Example 17. Preparation of S-3-hydroxybutyric acid·potassium S-3-hydroxybutyrate Complex (Complex I)

60 g of methyl S-3-hydroxybutyrate, 360 mL water and 24 g of catalyst were added into a 1-L reaction bottle, heated at 90-95° C. for 24 hours until the reaction was complete. It was cooled to room temperature, and the catalyst was filtered off. 50 mL aqueous solution of 13 g of potassium hydroxide was added to the filtrate, and the water was distilled off under reduced pressure. After evaporation to near dryness, 100 mL of dichloromethane was added. It was stirred, cooled to 0-10° C.; the solid was precipitated, filtered, and dried at 40° C., to obtain 48 g of S-3-hydroxybutyric acid·potassium S-3-hydroxybutyrate complex.

### Example 18. Preparation of S-3-hydroxybutyric acid·potassium S-3-hydroxybutyrate Complex (Complex I)

60 g of methyl S-3-hydroxybutyrate, 360 mL water and 24 g of catalyst were added into a 1-L reaction bottle, heated at 90-95° C. for 24 hours until the reaction was complete. It was cooled to room temperature, and the catalyst was filtered off. 50 mL aqueous solution of 13 g of potassium hydroxide was added to the filtrate, and the water was distilled off under reduced pressure. After evaporation to near dryness, 100 mL of acetone was added. It was stirred, cooled to 0-10° C.; the solid was precipitated, filtered, and dried at 55° C., to obtain 55 g of S-3-hydroxybutyric acid·potassium S-3-hydroxybutyrate complex.

### Example 19. Preparation of R-3-hydroxybutyric acid·calcium R-3-hydroxybutyrate Complex (Complex II)

100 g of R-3-hydroxybutyric acid, 120 g of calcium R-3-hydroxybutyrate, 440 mL dichloromethane were added into a 1-L reaction bottle, heated to 40° C. It was stirred and dissolved, cooled to 0-10° C.; the solid was precipitated, filtered, and dried at 40° C., to obtain 150 g of R-3-hydroxybutyric acid·calcium R-3-hydroxybutyrate complex.

### Example 20. Preparation of R-3-hydroxybutyric acid·calcium R-3-hydroxybutyrate Complex (Complex II)

104 g of R-3-hydroxybutyric acid, 125 g of calcium R-3-hydroxybutyrate, 480 mL acetone were added into a 1-L reaction bottle, heated to 60° C. It was stirred and dissolved, cooled to 0-10° C.; the solid was precipitated, filtered, and dried at 55° C., to obtain 140 g of R-3-hydroxybutyric acid·calcium R-3-hydroxybutyrate complex.

### Example 21. Preparation of R-3-hydroxybutyric acid·calcium R-3-hydroxybutyrate Complex (Complex II)

200 mL water and 14 g calcium oxide were added into a 1-L reaction bottle. It was stirred, heated to 55° C., and then 104 g of R-3-hydroxybutyric acid was added. It was stirred for half an hour, and distilled under reduced pressure to remove water. After evaporation to near dryness, 200 mL of dichloromethane was added. It was stirred, cooled to 0-10° C.; the solid was precipitated, filtered, and dried at 40° C., to obtain 65 g of R-3-hydroxybutyric acid·calcium R-3-hydroxybutyrate complex.

### Example 22. Preparation of R-3-hydroxybutyric acid·calcium R-3-hydroxybutyrate Complex (Complex II)

200 mL water and 14 g calcium oxide were added into a 1-L reaction bottle. It was stirred, heated to 55° C., and then 104 g of R-3-hydroxybutyric acid was added. It was stirred for half an hour, and distilled under reduced pressure to remove water. After evaporation to near dryness, 200 mL of acetone was added. It was stirred, cooled to 0-10° C.; the solid was precipitated, filtered, and dried at 55° C., to obtain 88 g of R-3-hydroxybutyric acid·calcium R-3-hydroxybutyrate complex.

### Example 23. Preparation of R-3-hydroxybutyric acid·calcium R-3-hydroxybutyrate Complex (Complex II)

60 g of methyl R-3-hydroxybutyrate, 360 mL water and 24 g of catalyst were added into a 1-L reaction bottle, heated at 90-95° C. for 24 hours until the reaction was complete. It was cooled to room temperature, and the catalyst was filtered off. 7 g of calcium oxide solid was added to the filtrate, and the water was distilled off under reduced pressure. After evaporation to near dryness, 100 mL of dichloromethane was added. It was stirred, cooled to 0-10° C.; the solid was precipitated, filtered, and dried at 40° C., to obtain 32 g of R-3-hydroxybutyric acid·calcium R-3-hydroxybutyrate complex.

### Example 24. Preparation of R-3-hydroxybutyric acid·calcium R-3-hydroxybutyrate Complex (Complex II)

60 g of methyl R-3-hydroxybutyrate, 360 mL water and 24 g of catalyst were added into a 1-L reaction bottle, heated at 90-95° C. for 24 hours until the reaction was complete. It was cooled to room temperature, and the catalyst was filtered off. 7 g of calcium oxide solid was added to the filtrate, and the water was distilled off under reduced pressure. After evaporation to near dryness, 100 mL of acetone was added. It was stirred, cooled to 0-10° C.; the solid was precipitated, filtered, and dried at 55° C., to obtain 35 g of R-3-hydroxybutyric acid·calcium R-3-hydroxybutyrate complex.

### Example 25. Preparation of 3-hydroxybutyric acid·calcium 3-hydroxybutyrate Complex (Complex II)

100 g of 3-hydroxybutyric acid, 120 g of calcium 3-hydroxybutyrate, 440 mL dichloromethane were added into a 1-L reaction bottle, heated to 40° C. It was stirred and dissolved, cooled to 0-10° C.; the solid was precipitated, filtered, and dried at 40° C., to obtain 143 g of 3-hydroxybutyric acid·calcium 3-hydroxybutyrate complex.

### Example 26. Preparation of 3-hydroxybutyric acid·calcium 3-hydroxybutyrate Complex (Complex II)

104 g of 3-hydroxybutyric acid, 125 g of calcium 3-hydroxybutyrate, 480 mL acetone were added into a 1-L reaction bottle, heated to 60° C. It was stirred and dissolved, cooled to 0-10° C.; the solid was precipitated, filtered, and dried at 55° C., to obtain 132 g of 3-hydroxybutyric acid·calcium 3-hydroxybutyrate complex.

### Example 27. Preparation of 3-hydroxybutyric acid·calcium 3-hydroxybutyrate Complex (Complex II)

200 mL water and 14 g calcium oxide were added into a 1-L reaction bottle. It was stirred, heated to 55° C., and then 104 g of 3-hydroxybutyric acid was added. It was stirred for half an hour, and distilled under reduced pressure to remove water. After evaporation to near dryness, 200 mL of dichloromethane was added. It was stirred, cooled to 0-10° C.; the solid was precipitated, filtered, and dried at 40° C., to obtain 69 g of 3-hydroxybutyric acid·calcium 3-hydroxybutyrate complex.

### Example 28. Preparation of 3-hydroxybutyric acid·calcium 3-hydroxybutyrate Complex (Complex II)

200 mL water and 14 g calcium oxide were added into a 1-L reaction bottle. It was stirred, heated to 55° C., and then 104 g of 3-hydroxybutyric acid was added. It was stirred for half an hour, and distilled under reduced pressure to remove water. After evaporation to near dryness, 200 mL of acetone was added. It was stirred, cooled to 0-10° C.; the solid was precipitated, filtered, and dried at 55° C., to obtain 85 g of 3-hydroxybutyric acid·calcium 3-hydroxybutyrate complex.

### Example 29. Preparation of 3-hydroxybutyric acid·calcium 3-hydroxybutyrate Complex (Complex II)

60 g of methyl 3-hydroxybutyrate, 360 mL water and 24 g of catalyst were added into a 1-L reaction bottle, heated at 90-95° C. for 24 hours until the reaction was complete. It was cooled to room temperature, and the catalyst was filtered off. 7 g of calcium oxide solid was added to the filtrate, and the water was distilled off under reduced pressure. After evaporation to near dryness, 100 mL of dichloromethane was added. It was stirred, cooled to 0-10° C.; the solid was precipitated, filtered, and dried at 40° C., to obtain 37 g of 3-hydroxybutyric acid·calcium 3-hydroxybutyrate complex.

### Example 30. Preparation of 3-hydroxybutyric acid·calcium 3-hydroxybutyrate Complex (Complex II)

60 g of methyl 3-hydroxybutyrate, 360 mL water and 24 g of catalyst were added into a 1-L reaction bottle, heated at 90-95° C. for 24 hours until the reaction was complete. It was cooled to room temperature, and the catalyst was filtered off. 7 g of calcium oxide solid was added to the filtrate, and the water was distilled off under reduced pressure. After evaporation to near dryness, 100 mL of acetone was added. It was stirred, cooled to 0-10° C.; the solid was precipitated, filtered, and dried at 55° C., to obtain 34 g of 3-hydroxybutyric acid·calcium 3-hydroxybutyrate complex.

### Example 31. Preparation of S-3-hydroxybutyric acid·calcium S-3-hydroxybutyrate Complex (Complex II)

100 g of S-3-hydroxybutyric acid, 120 g of calcium S-3-hydroxybutyrate, 440 mL dichloromethane were added into a 1-L reaction bottle, heated to 40° C. It was stirred and dissolved, cooled to 0-10° C.; the solid was precipitated, filtered, and dried at 40° C., to obtain 148 g of S-3-hydroxybutyric acid·calcium S-3-hydroxybutyrate complex.

### Example 32. Preparation of S-3-hydroxybutyric acid·calcium S-3-hydroxybutyrate Complex (Complex II)

104 g of S-3-hydroxybutyric acid, 125 g of calcium S-3-hydroxybutyrate, 480 mL acetone were added into a 1-L reaction bottle, heated to 60° C. It was stirred and dissolved, cooled to 0-10° C.; the solid was precipitated, filtered, and dried at 55° C., to obtain 143 g of S-3-hydroxybutyric acid·calcium S-3-hydroxybutyrate complex.

### Example 33. Preparation of S-3-hydroxybutyric acid·calcium S-3-hydroxybutyrate Complex (Complex II)

200 mL water and 14 g calcium oxide were added into a 1-L reaction bottle. It was stirred, heated to 55° C., and then 104 g of S-3-hydroxybutyric acid was added. It was stirred for half an hour, and distilled under reduced pressure to remove water. After evaporation to near dryness, 200 mL of dichloromethane was added. It was stirred, cooled to 0-10° C.; the solid was precipitated, filtered, and dried at 40° C., to obtain 59 g of S-3-hydroxybutyric acid·calcium S-3-hydroxybutyrate complex.

### Example 34. Preparation of S-3-hydroxybutyric acid·calcium S-3-hydroxybutyrate Complex (Complex II)

200 mL water and 14 g calcium oxide were added into a 1-L reaction bottle. It was stirred, heated to 55° C., and then 104 g of S-3-hydroxybutyric acid was added. It was stirred for half an hour, and distilled under reduced pressure to remove water. After evaporation to near dryness, 200 mL of acetone was added. It was stirred, cooled to 0-10° C.; the solid was precipitated, filtered, and dried at 55° C., to obtain 83 g of S-3-hydroxybutyric acid·calcium S-3-hydroxybutyrate complex.

### Example 35. Preparation of S-3-hydroxybutyric acid·calcium S-3-hydroxybutyrate Complex (Complex II)

60 g of methyl S-3-hydroxybutyrate, 360 mL water and 24 g of catalyst were added into a 1-L reaction bottle, heated at 90-95° C. for 24 hours until the reaction was complete. It was cooled to room temperature, and the catalyst was filtered off. 7 g of calcium oxide solid was added to the filtrate, and the water was distilled off under reduced pressure. After evaporation to near dryness, 100 mL of dichloromethane was added. It was stirred, cooled to 0-10° C.; the solid was precipitated, filtered, and dried at 40° C., to obtain 30 g of S-3-hydroxybutyric acid·calcium S-3-hydroxybutyrate complex.

### Example 36. Preparation of S-3-hydroxybutyric acid·calcium S-3-hydroxybutyrate Complex (Complex II)

60 g of methyl S-3-hydroxybutyrate, 360 mL water and 24 g of catalyst were added into a 1-L reaction bottle, heated at 90-95° C. for 24 hours until the reaction was complete. It was cooled to room temperature, and the catalyst was filtered off. 7 g of calcium oxide solid was added to the filtrate, and the water was distilled off under reduced pressure. After evaporation to near dryness, 100 mL of acetone was added. It was stirred, cooled to 0-10° C.; the solid was precipitated, filtered, and dried at 55° C., to obtain 32 g of S-3-hydroxybutyric acid·calcium S-3-hydroxybutyrate complex.

### Example 37. Preparation of R-3-hydroxybutyric acid·magnesium R-3-hydroxybutyrate Complex (Complex III)

100 g of R-3-hydroxybutyric acid, 110 g of magnesium R-3-hydroxybutyrate, 440 mL dichloromethane were added into a 1-L reaction bottle, heated to 40° C. It was stirred and dissolved, cooled to 0-10° C.; the solid was precipitated, filtered, and dried at 40° C., to obtain 148 g of R-3-hydroxybutyric acid·magnesium R-3-hydroxybutyrate complex.

### Example 38. Preparation of R-3-hydroxybutyric acid·magnesium R-3-hydroxybutyrate Complex (Complex III)

104 g of R-3-hydroxybutyric acid, 116 g of magnesium R-3-hydroxybutyrate, 480 mL acetone were added into a 1-L reaction bottle, heated to 60° C. It was stirred and dissolved, cooled to 0-10° C.; the solid was precipitated, filtered, and dried at 55° C., to obtain 140 g of R-3-hydroxybutyric acid·magnesium R-3-hydroxybutyrate complex.

### Example 39. Preparation of R-3-hydroxybutyric acid·magnesium R-3-hydroxybutyrate Complex (Complex III)

200 mL water and 12 g magnesium oxide were added into a 1-L reaction bottle. It was stirred, heated to 55° C., and then 104 g of R-3-hydroxybutyric acid was added. It was stirred for half an hour, and distilled under reduced pressure to remove water. After evaporation to near dryness, 200 mL of dichloromethane was added. It was stirred, cooled to 0-10° C.; the solid was precipitated, filtered, and dried at 40° C., to obtain 62 g of R-3-hydroxybutyric acid·magnesium R-3-hydroxybutyrate complex.

### Example 40. Preparation of R-3-hydroxybutyric acid·magnesium R-3-hydroxybutyrate Complex (Complex III)

200 mL water and 12 g magnesium oxide were added into a 1-L reaction bottle. It was stirred, heated to 55° C., and then 104 g of R-3-hydroxybutyric acid was added. It was stirred for half an hour, and distilled under reduced pressure to remove water. After evaporation to near dryness, 200 mL of acetone was added. It was stirred, cooled to 0-10° C.; the solid was precipitated, filtered, and dried at 55° C., to obtain 82 g of R-3-hydroxybutyric acid·magnesium R-3-hydroxybutyrate complex.

### Example 41. Preparation of R-3-hydroxybutyric acid·magnesium R-3-hydroxybutyrate Complex (Complex III)

60 g of methyl R-3-hydroxybutyrate, 360 mL water and 24 g of catalyst were added into a 1-L reaction bottle, heated at 90-95° C. for 24 hours until the reaction was complete. It was cooled to room temperature, and the catalyst was filtered off. 7 g of magnesium oxide solid was added to the filtrate, and the water was distilled off under reduced pressure. After evaporation to near dryness, 100 mL of dichloromethane was added. It was stirred, cooled to 0-10° C.; the solid was precipitated, filtered, and dried at 40° C., to obtain 32 g of R-3-hydroxybutyric acid·magnesium R-3-hydroxybutyrate complex.

### Example 42. Preparation of R-3-hydroxybutyric acid·magnesium R-3-hydroxybutyrate Complex (Complex III)

60 g of methyl R-3-hydroxybutyrate, 360 mL water and 24 g of catalyst were added into a 1-L reaction bottle, heated at 90-95° C. for 24 hours until the reaction was complete. It was cooled to room temperature, and the catalyst was filtered off. 7 g of magnesium oxide solid was added to the filtrate, and the water was distilled off under reduced pressure. After evaporation to near dryness, 100 mL of acetone was added. It was stirred, cooled to 0-10° C.; the solid was precipitated, filtered, and dried at 55° C., to obtain 35 g of R-3-hydroxybutyric acid·magnesium R-3-hydroxybutyrate complex.

### Example 43. Preparation of 3-hydroxybutyric acid·magnesium 3-hydroxybutyrate Complex (Complex III)

100 g of 3-hydroxybutyric acid, 110 g of magnesium 3-hydroxybutyrate, 440 mL dichloromethane were added into a 1-L reaction bottle, heated to 40° C. It was stirred and dissolved, cooled to 0-10° C.; the solid was precipitated, filtered, and dried at 40° C., to obtain 145 g of 3-hydroxybutyric acid·magnesium 3-hydroxybutyrate complex.

### Example 44. Preparation of 3-hydroxybutyric acid·magnesium 3-hydroxybutyrate Complex (Complex III)

104 g of 3-hydroxybutyric acid, 116 g of magnesium 3-hydroxybutyrate, 480 mL acetone were added into a 1-L reaction bottle, heated to 60° C. It was stirred and dissolved, cooled to 0-10° C.; the solid was precipitated, filtered, and dried at 55° C., to obtain 148 g of 3-hydroxybutyric acid·magnesium 3-hydroxybutyrate complex.

### Example 45. Preparation of 3-hydroxybutyric acid·magnesium 3-hydroxybutyrate Complex (Complex III)

200 mL water and 12 g magnesium oxide were added into a 1-L reaction bottle. It was stirred, heated to 55° C., and then 104 g of 3-hydroxybutyric acid was added. It was stirred for half an hour, and distilled under reduced pressure to remove water. After evaporation to near dryness, 200 mL of dichloromethane was added. It was stirred, cooled to 0-10° C.; the solid was precipitated, filtered, and dried at 40° C., to obtain 67 g of 3-hydroxybutyric acid·magnesium 3-hydroxybutyrate complex.

### Example 46. Preparation of 3-hydroxybutyric acid·magnesium 3-hydroxybutyrate Complex (Complex III)

200 mL water and 12 g magnesium oxide were added into a 1-L reaction bottle. It was stirred, heated to 55° C., and then 104 g of 3-hydroxybutyric acid was added. It was stirred for half an hour, and distilled under reduced pressure to remove water. After evaporation to near dryness, 200 mL of acetone was added. It was stirred, cooled to 0-10° C.; the solid was precipitated, filtered, and dried at 55° C., to obtain 76 g of 3-hydroxybutyric acid·magnesium 3-hydroxybutyrate complex.

### Example 47. Preparation of 3-hydroxybutyric acid·magnesium 3-hydroxybutyrate Complex (Complex III)

60 g of methyl 3-hydroxybutyrate, 360 mL water and 24 g of catalyst were added into a 1-L reaction bottle, heated at 90-95° C. for 24 hours until the reaction was complete. It was cooled to room temperature, and the catalyst was filtered off. 7 g of magnesium oxide solid was added to the filtrate, and the water was distilled off under reduced pressure. After evaporation to near dryness, 100 mL of dichloromethane was added. It was stirred, cooled to 0-10° C.; the solid was precipitated, filtered, and dried at 40° C., to obtain 35 g of 3-hydroxybutyric acid·magnesium 3-hydroxy butyrate complex.

### Example 48. Preparation of 3-hydroxybutyric acid·magnesium 3-hydroxybutyrate Complex (Complex III)

60 g of methyl 3-hydroxybutyrate, 360 mL water and 24 g of catalyst were added into a 1-L reaction bottle, heated at 90-95° C. for 24 hours until the reaction was complete. It was cooled to room temperature, and the catalyst was filtered off. 7 g of magnesium oxide solid was added to the filtrate, and the water was distilled off under reduced pressure. After evaporation to near dryness, 100 mL of acetone was added. It was stirred, cooled to 0-10° C.; the solid was precipitated, filtered, and dried at 55° C., to obtain 36 g of 3-hydroxybutyric acid·magnesium 3-hydroxybutyrate complex.

### Example 49. Preparation of S-3-hydroxybutyric acid·magnesium S-3-hydroxybutyrate Complex (Complex III)

100 g of S-3-hydroxybutyric acid, 110 g of magnesium S-3-hydroxybutyrate, 440 mL dichloromethane were added into a 1-L reaction bottle, heated to 40° C. It was stirred and dissolved, cooled to 0-10° C.; the solid was precipitated, filtered, and dried at 40° C., to obtain 145 g of S-3-hydroxybutyric acid·magnesium S-3-hydroxybutyrate complex.

### Example 50. Preparation of S-3-hydroxybutyric acid·magnesium S-3-hydroxybutyrate Complex (Complex III)

104 g of S-3-hydroxybutyric acid, 116 g of magnesium S-3-hydroxybutyrate, 480 mL acetone were added into a 1-L reaction bottle, heated to 60° C. It was stirred and dissolved, cooled to 0-10° C.; the solid was precipitated, filtered, and dried at 55° C., to obtain 138 g of S-3-hydroxybutyric acid·magnesium S-3-hydroxybutyrate complex.

### Example 51. Preparation of S-3-hydroxybutyric acid·magnesium S-3-hydroxybutyrate Complex (Complex III)

200 mL water and 12 g magnesium oxide were added into a 1-L reaction bottle. It was stirred, heated to 55° C., and then 104 g of S-3-hydroxybutyric acid was added. It was stirred for half an hour, and distilled under reduced pressure to remove water. After evaporation to near dryness, 200 mL of dichloromethane was added. It was stirred, cooled to 0-10° C.; the solid was precipitated, filtered, and dried at 40° C., to obtain 58 g of S-3-hydroxybutyric acid·magnesium S-3-hydroxybutyrate complex.

### Example 52. Preparation of S-3-hydroxybutyric acid·magnesium S-3-hydroxybutyrate Complex (Complex III)

200 mL water and 12 g magnesium oxide were added into a 1-L reaction bottle. It was stirred, heated to 55° C., and then 104 g of S-3-hydroxybutyric acid was added. It was stirred for half an hour, and distilled under reduced pressure to remove water. After evaporation to near dryness, 200 mL of acetone was added. It was stirred, cooled to 0-10° C.; the solid was precipitated, filtered, and dried at 55° C., to obtain 83 g of S-3-hydroxybutyric acid·magnesium S-3-hydroxybutyrate complex.

### Example 53. Preparation of S-3-hydroxybutyric acid·magnesium S-3-hydroxybutyrate Complex (Complex III)

60 g of methyl S-3-hydroxybutyrate, 360 mL water and 24 g of catalyst were added into a 1-L reaction bottle, heated at 90-95° C. for 24 hours until the reaction was complete. It was cooled to room temperature, and the catalyst was filtered off. 7 g of magnesium oxide solid was added to the filtrate, and the water was distilled off under reduced pressure. After evaporation to near dryness, 100 mL of dichloromethane was added. It was stirred, cooled to 0-10° C.; the solid was precipitated, filtered, and dried at 40° C., to obtain 34 g of S-3-hydroxybutyric acid·magnesium S-3-hydroxybutyrate complex.

### Example 54. Preparation of S-3-hydroxybutyric acid·magnesium S-3-hydroxybutyrate Complex (Complex III)

60 g of methyl S-3-hydroxybutyrate, 360 mL water and 24 g of catalyst were added into a 1-L reaction bottle, heated at 90-95° C. for 24 hours until the reaction was complete. It was cooled to room temperature, and the catalyst was filtered off. 7 g of magnesium oxide solid was added to the filtrate, and the water was distilled off under reduced pressure. After evaporation to near dryness, 100 mL of acetone was added. It was stirred, cooled to 0-10° C.; the solid was precipitated, filtered, and dried at 55° C., to obtain 36 g of S-3-hydroxybutyric acid·magnesium S-3-hydroxybutyrate complex.

### Example 55. Preparation of R-3-hydroxypentanoic acid·sodium R-3-hydroxypentanoate Complex (Complex IV)

118 g of R-3-hydroxypentanoic acid, 140 g of sodium R-3-hydroxypentanoate, 450 mL dichloromethane were added into a 1-L reaction bottle, heated to 40° C. It was stirred and dissolved, cooled to 0-10° C.; the solid was precipitated, filtered, and dried at 40° C., to obtain 148 g of R-3-hydroxypentanoic acid·sodium R-3-hydroxypentanoate complex.

### Example 56. Preparation of R-3-hydroxypentanoic acid sodium R-3-hydroxypentanoate Complex (Complex IV)

118 g of R-3-hydroxypentanoic acid, 140 g of sodium R-3-hydroxypentanoate, 480 mL acetone were added into a 1-L reaction bottle, heated to 60° C. It was stirred and dissolved, cooled to 0-10° C.; the solid was precipitated, filtered, and dried at 55° C., to obtain 140 g of R-3-hydroxypentanoic acid·sodium R-3-hydroxypentanoate complex.

### Example 57. Preparation of R-3-hydroxypentanoic acid·sodium R-3-hydroxypentanoate Complex (Complex IV)

200 mL water and 20 g sodium hydroxide were added into a 1-L reaction bottle. It was stirred and dissolved, cooled to below 25° C., and then 120 g of R-3-hydroxypentanoic acid was added. It was stirred for half an hour, and distilled under reduced pressure to remove water. After evaporation to near dryness, 200 ml of dichloromethane was added. It was stirred, cooled to 0-10° C.; the solid was precipitated, filtered, and dried at 40° C., to obtain 75 g of R-3-hydroxypentanoic acid·sodium R-3-hydroxypentanoate complex.

### Example 58. Preparation of R-3-hydroxypentanoic acid sodium R-3-hydroxypentanoate Complex (Complex IV)

200 mL water and 20 g sodium hydroxide were added into a 1-L reaction bottle. It was stirred and dissolved, cooled to below 25° C., and then 120 g of R-3-hydroxypentanoic acid was added. It was stirred for half an hour, and distilled under reduced pressure to remove water. After evaporation to near dryness, 200 mL of acetone was added. It was stirred, cooled to 0-10° C.; the solid was precipitated, filtered, and dried at 55° C., to obtain 98 g of R-3-hydroxypentanoic acid·sodium R-3-hydroxypentanoate complex.

### Example 59. Preparation of R-3-hydroxypentanoic acid·sodium R-3-hydroxypentanoate Complex (Complex IV)

65 g of methyl R-3-hydroxypentanoate, 360 mL water and 24 g of catalyst were added into a 1-L reaction bottle, heated at 90-95° C. for 24 hours until the reaction was complete. It was cooled to room temperature, and the catalyst was filtered off. 50 mL aqueous solution of 9 g of sodium hydroxide was added to the filtrate, and the water was distilled off under reduced pressure. After evaporation to near dryness, 100 mL of dichloromethane was added. It was stirred, cooled to 0-10° C.; the solid was precipitated, filtered, and dried at 40° C., to obtain 40 g of R-3-hydroxypentanoic acid·sodium R-3-hydroxypentanoate complex.

### Example 60. Preparation of R-3-hydroxypentanoic acid·sodium R-3-hydroxypentanoate Complex (Complex IV)

65 g of methyl R-3-hydroxypentanoate, 360 mL water and 24 g of catalyst were added into a 1-L reaction bottle, heated at 90-95° C. for 24 hours until the reaction was complete. It was cooled to room temperature, and the catalyst was filtered off. 50 mL aqueous solution of 9 g of sodium hydroxide was added to the filtrate, and the water was distilled off under reduced pressure. After evaporation to near dryness, 100 mL of acetone was added. It was stirred, cooled to 0-10° C.; the solid was precipitated, filtered, and dried at 55° C., to obtain 50 g of R-3-hydroxypentanoic acid·sodium R-3-hydroxypentanoate complex.

### Example 61. Preparation of 3-hydroxypentanoic acid·sodium 3-hydroxypentanoate Complex (Complex IV)

118 g of 3-hydroxypentanoic acid, 140 g of sodium 3-hydroxypentanoate, 450 mL dichloromethane were added into a 1-L reaction bottle, heated to 40° C. It was stirred and dissolved, cooled to 0-10° C.; the solid was precipitated, filtered, and dried at 40° C., to obtain 139 g of 3-hydroxypentanoic acid·sodium 3-hydroxypentanoate complex.

### Example 62. Preparation of 3-hydroxypentanoic acid·sodium 3-hydroxypentanoate Complex (Complex IV)

118 g of 3-hydroxypentanoic acid, 140 g of sodium 3-hydroxypentanoate, 480 mL acetone were added into a 1-L reaction bottle, heated to 60° C. It was stirred and dissolved, cooled to 0-10° C.; the solid was precipitated, filtered, and dried at 55° C., to obtain 124 g of 3-hydroxypentanoic acid·sodium 3-hydroxypentanoate complex.

### Example 63. Preparation of 3-hydroxypentanoic acid·sodium 3-hydroxypentanoate Complex (Complex IV)

200 mL water and 20 g sodium hydroxide were added into a 1-L reaction bottle. It was stirred and dissolved, cooled to below 25° C., and then 120 g of 3-hydroxypentanoic acid was added. It was stirred for half an hour, and distilled under reduced pressure to remove water. After evaporation to near dryness, 200 mL of dichloromethane was added. It was stirred, cooled to 0-10° C.; the solid was precipitated, filtered, and dried at 40° C., to obtain 72 g of 3-hydroxypentanoic acid·sodium 3-hydroxypentanoate complex.

### Example 64. Preparation of 3-hydroxypentanoic acid·sodium 3-hydroxypentanoate Complex (Complex IV)

200 mL water and 20 g sodium hydroxide were added into a 1-L reaction bottle. It was stirred and dissolved, cooled to below 25° C., and then 120 g of 3-hydroxypentanoic acid was added. It was stirred for half an hour, and distilled under reduced pressure to remove water. After evaporation to near dryness, 200 mL of acetone was added. It was stirred, cooled to 0-10° C.; the solid was precipitated, filtered, and dried at 55° C., to obtain 68 g of 3-hydroxypentanoic acid·sodium 3-hydroxypentanoate complex.

### Example 65. Preparation of 3-hydroxypentanoic acid·sodium 3-hydroxypentanoate Complex (Complex IV)

65 g of methyl 3-hydroxypentanoate, 360 mL water and 24 g of catalyst were added into a 1-L reaction bottle, heated at 90-95° C. for 24 hours until the reaction was complete. It was cooled to room temperature, and the catalyst was filtered off. 50 mL aqueous solution of 9 g of sodium hydroxide was added to the filtrate, and the water was distilled off under reduced pressure. After evaporation to near dryness, 100 mL of dichloromethane was added. It was stirred, cooled to 0-10° C.; the solid was precipitated, filtered, and dried at 40° C., to obtain 45 g of 3-hydroxypentanoic acid·sodium 3-hydroxypentanoate complex.

### Example 66. Preparation of 3-hydroxypentanoic acid·sodium 3-hydroxypentanoate Complex (Complex IV)

65 g of methyl 3-hydroxypentanoate, 360 mL water and 24 g of catalyst were added into a 1-L reaction bottle, heated at 90-95° C. for 24 hours until the reaction was complete. It was cooled to room temperature, and the catalyst was filtered off. 50 mL aqueous solution of 9 g of sodium hydroxide was added to the filtrate, and the water was distilled off under reduced pressure. After evaporation to near dryness, 100 mL of acetone was added. It was stirred, cooled to 0-10° C.; the solid was precipitated, filtered, and dried at 55° C., to obtain 46 g of 3-hydroxypentanoic acid·sodium 3-hydroxypentanoate complex.

### Example 67. Preparation of S-3-hydroxypentanoic acid·sodium S-3-hydroxypentanoate Complex (Complex IV)

118 g of S-3-hydroxypentanoic acid, 140 g of sodium S-3-hydroxypentanoate, 450 mL dichloromethane were added into a 1-L reaction bottle, heated to 40° C. It was stirred and dissolved, cooled to 0-10° C.; the solid was precipitated, filtered, and dried at 40° C., to obtain 147 g of S-3-hydroxypentanoic acid·sodium S-3-hydroxypentanoate complex.

### Example 68. Preparation of S-3-hydroxypentanoic acid·sodium S-3-hydroxypentanoate Complex (Complex IV)

118 g of S-3-hydroxypentanoic acid, 140 g of sodium S-3-hydroxypentanoate, 480 mL acetone were added into a 1-L reaction bottle, heated to 60° C. It was stirred and dissolved, cooled to 0-10° C.; the solid was precipitated, filtered, and dried at 55° C., to obtain 138 g of S-3-hydroxypentanoic acid·sodium S-3-hydroxypentanoate complex.

### Example 69. Preparation of S-3-hydroxypentanoic acid·sodium S-3-hydroxypentanoate Complex (Complex IV)

200 mL water and 20 g sodium hydroxide were added into a 1-L reaction bottle. It was stirred and dissolved, cooled to below 25° C., and then 120 g of S-3-hydroxypentanoic acid was added. It was stirred for half an hour, and distilled under reduced pressure to remove water. After evaporation to near dryness, 200 mL of dichloromethane was added. It was stirred, cooled to 0-10° C.; the solid was precipitated, filtered, and dried at 40° C., to obtain 68 g of S-3-hydroxypentanoic acid·sodium S-3-hydroxypentanoate complex.

### Example 70. Preparation of S-3-hydroxypentanoic acid sodium S-3-hydroxypentanoate Complex (Complex IV)

200 mL water and 20 g sodium hydroxide were added into a 1-L reaction bottle. It was stirred and dissolved, cooled to below 25° C., and then 120 g of S-3-hydroxypentanoic acid was added. It was stirred for half an hour, and distilled under reduced pressure to remove water. After evaporation to near dryness, 200 mL of acetone was added. It was stirred, cooled to 0-10° C.; the solid was precipitated, filtered, and dried at 55° C., to obtain 92 g of S-3-hydroxypentanoic acid·sodium S-3-hydroxypentanoate complex.

### Example 71. Preparation of S-3-hydroxypentanoic acid·sodium S-3-hydroxypentanoate Complex (Complex IV)

65 g of methyl S-3-hydroxypentanoate, 360 mL water and 24 g of catalyst were added into a 1-L reaction bottle, heated at 90-95° C. for 24 hours until the reaction was complete. It was cooled to room temperature, and the catalyst was filtered off. 50 mL aqueous solution of 9 g of sodium hydroxide was added to the filtrate, and the water was distilled off under reduced pressure. After evaporation to near dryness, 100 mL of dichloromethane was added. It was stirred, cooled to 0-10° C.; the solid was precipitated, filtered, and dried at 40° C., to obtain 41 g of S-3-hydroxypentanoic acid·sodium S-3-hydroxypentanoate complex.

### Example 72. Preparation of S-3-hydroxypentanoic acid·sodium S-3-hydroxypentanoate Complex (Complex IV)

65 g of methyl S-3-hydroxypentanoate, 360 mL water and 24 g of catalyst were added into a 1-L reaction bottle, heated at 90-95° C. for 24 hours until the reaction was complete. It was cooled to room temperature, and the catalyst was filtered off. 50 mL aqueous solution of 9 g of sodium hydroxide was added to the filtrate, and the water was distilled off under reduced pressure. After evaporation to near dryness, 100 mL of acetone was added. It was stirred, cooled to 0-10° C.; the solid was precipitated, filtered, and dried at 55° C., to obtain 48 g of S-3-hydroxypentanoic acid·sodium S-3-hydroxypentanoate complex.

The acid-sodium salt complex, acid-potassium salt complex, acid-calcium salt complex and acid-magnesium salt complex of the present invention are prepared by basically the same method as complexes I to IV.

### Characterization of Complexes of the Present Invention

X-ray diffraction (XRD), elemental analysis, Raman spectroscopy, infrared spectroscopy (IR), TGA, DSC, DVS, etc. were performed on the complexes prepared in the Examples.

### Example 73. X-Ray Diffraction

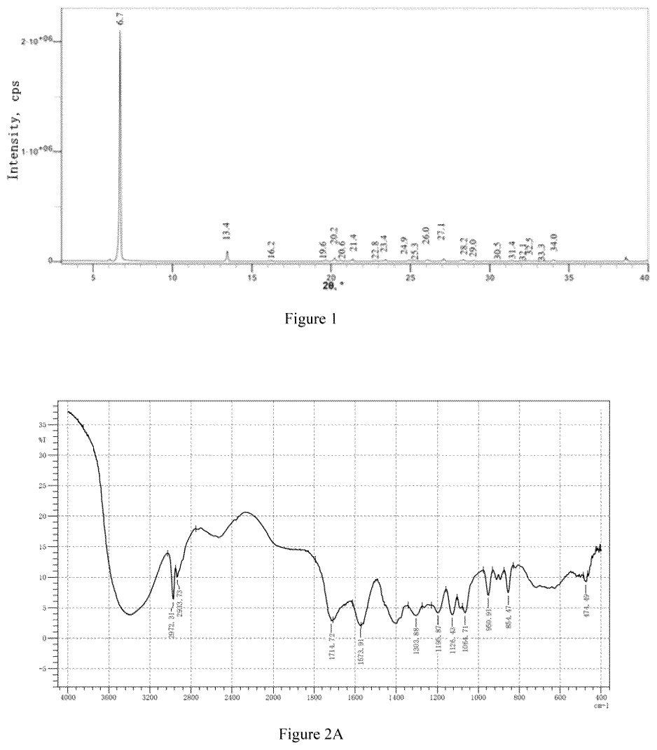

X-ray powder diffraction patterns were obtained by using SmartLab 3KW X-ray powder diffractometer under the following conditions: diffraction line: Cu K-beta (40 KV, 40 mA), scanning rate: 20.00 deg/min, and scanning range: 3° ˜60°. The XRPD pattern of the 3-hydroxybutyric acid·potassium 3-hydroxybutyrate complex (Complex I) obtained in Example 1 is shown in FIG. 1, and the XRPD data is shown in Table 1.

TABLE 1

| | | relative intensity |
|---|---|---|
| position (2θ, °) | spacing (d, Å) | (%) |
| 6.7 | 13.2 | 100 |
| 13.4 | 6.6 | 5.04 |
| 16.2 | 5.5 | 0.27 |
| 19.6 | 4.5 | 0.83 |
| 20.2 | 4.4 | 1.97 |
| 20.6 | 4.3 | 0.48 |
| 21.4 | 4.2 | 1.07 |
| 22.8 | 3.9 | 0.37 |
| 23.4 | 3.8 | 1.08 |
| 24.9 | 3.6 | 0.81 |
| 25.3 | 3.5 | 0.3 |
| 26.0 | 3.4 | 1.37 |
| 27.1 | 3.3 | 1.54 |
| 28.2 | 3.2 | 0.64 |
| 29.0 | 3.1 | 0.47 |
| 31.4 | 2.8 | 0.37 |
| 32.1 | 2.8 | 0.36 |
| 32.5 | 2.8 | 0.52 |
| 33.3 | 2.7 | 0.18 |
| 34.0 | 2.6 | 0.93 |

The XRPD results of the complexes prepared in Examples 2-6 are basically the same as those in Example 1.

### Example 74. Infrared Spectroscopy

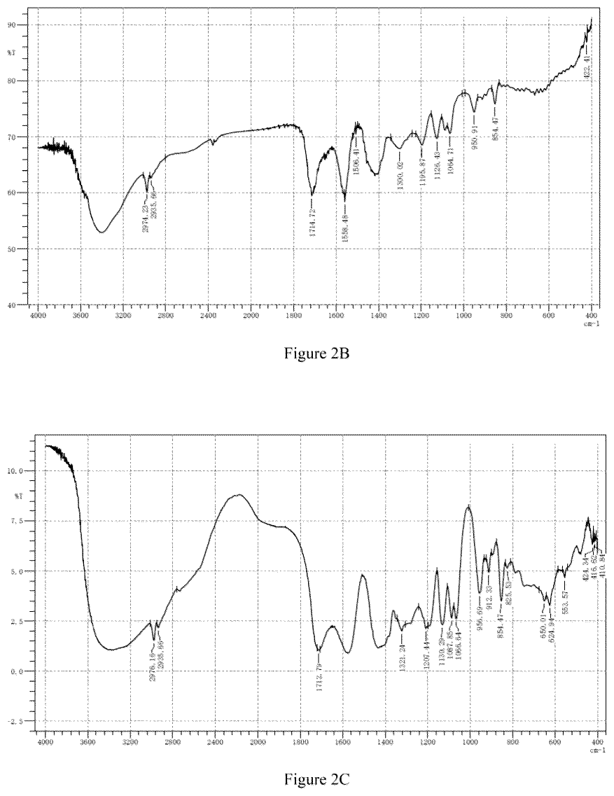

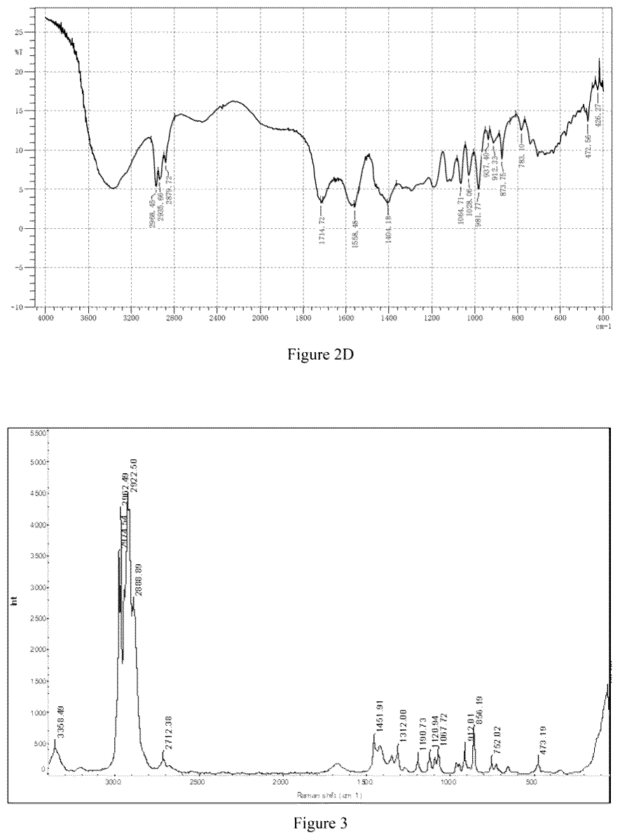

The infrared spectrum of the complex I of Example 1 was analyzed by SHIMADZU's Fourier transform attenuated total reflection infrared spectrometer. FIG. 2A shows an infrared spectrogram (IR) of the complex I of Example 1. As shown in FIG. 2A, the complex I has characteristic absorption peaks at 2972 cm⁻¹, 2933 cm⁻¹, 1715 cm⁻¹, 1574 cm⁻¹, 1304 cm⁻¹, 1196 cm⁻¹, 1126 cm⁻¹, 1065 cm⁻¹, 951 cm⁻¹, 854 cm 1, 474 cm⁻¹. The IR results of the complexes prepared in Examples 2-18 are substantially the same as those in Example 1. The infrared spectrum of the complex II of Example 19 was analyzed. FIG. 2B shows an infrared spectrogram (IR) of the complex II of Example 19. As shown in FIG. 2B, the complex II has characteristic absorption peaks at 2974 cm⁻¹, 2936 cm⁻¹, 1715 cm⁻¹, 1558 cm⁻¹, 1506 cm⁻¹, 1300 cm⁻¹, 1196 cm⁻¹, 1126 cm⁻¹, 1065 cm⁻¹, 951 cm⁻¹, 854 cm⁻¹, 422 cm⁻¹. The IR results of the complexes prepared in Examples 20-36 are substantially the same as those in Example 19. The infrared spectrum of the complex III of Example 37 was analyzed. FIG. 2C shows an infrared spectrogram (IR) of the complex III of Example 37. As shown in FIG. 2C, the complex III has characteristic absorption peaks at 2976 cm⁻¹, 2936 cm⁻¹, 1713 cm⁻¹, 1321 cm⁻¹, 1207 cm⁻¹, 1088 cm⁻¹, 957 cm⁻¹, 912 cm⁻¹, 826 cm⁻¹, 625 cm⁻¹, 554 cm⁻¹, 411 cm⁻¹. The IR results of the complexes prepared in Examples 38-54 are substantially the same as those in Example 37. The infrared spectrum of the complex IV of Example 55 was analyzed. FIG. 2D shows an infrared spectrogram (IR) of the complex IV of Example 55. As shown in FIG. 2D, the complex IV has characteristic absorption peaks at 2968 cm⁻¹, 2880 cm⁻¹, 1715 cm⁻¹, 1558 cm⁻¹, 1404 cm⁻¹, 1065 cm⁻¹, 982 cm⁻¹, 912 cm⁻¹, 874 cm⁻¹, 783 cm⁻¹, 473 cm⁻¹, 426 cm⁻¹. The IR results of the complexes prepared in Examples 56-72 are substantially the same as those in Example 55.

### Example 75. NMR (¹H/¹³C) Determination

¹H NMR and ¹³C NMR spectra of the Complex I of Example 1 were recorded in an AVIII-HD-400 spectrometer: ¹H NMR (400 MHz, DMSO-d6) δ 7.61 (s, 1H), 3.89 (dt, J=7.1, 5.9 Hz, 1H), 2.20-2.06 (m, 2H), 1.04 (d, J=6.2 Hz, 3H). ¹³C NMR (101 MHz, DMSO-d6) δ 175.58, 64.24, 45.69, 23.80. The NMR (¹H/¹³C) results of the Complex I prepared in Examples 2-18 are substantially the same as those in Example 1. ¹H NMR spectrum of the Complex II of Example 19 was recorded: ¹H NMR (400 MHz, D₂O) δ 4.06 (h, J=6.4 Hz, 1H), 2.32 (d, J=6.6 Hz, 2H), 1.09 (d, J=6.3 Hz, 3H). The NMR (1H) results of the Complex II prepared in Examples 20-36 and the Complex III prepared in Examples 37-54 are substantially the same as those in Example 19. ¹H NMR spectrum of the Complex IV of Example 55 was recorded: ¹H NMR (400 MHz, DMSO-d6) δ 3.68-3.53 (m, 1H), 2.17 (dd, J=14.8, 4.3 Hz, 1H), 2.03 (dd, J=14.8, 8.5 Hz, 1H), 1.40-1.26 (m, 2H), 0.84 (t, J=7.4 Hz, 3H). The NMR (1H) results of the Complex IV prepared in Examples 56-72 are substantially the same as those in Example 55.

### Example 76. Determination of Metal Ion Content and BHB/BHP Content

The potassium content in the complex I of Examples 1, 7 and 13, the calcium content in the complex II of Examples 19, 25 and 31, and the magnesium content in the complex III of Examples 37, 43 and 49 were measured; and the content of 3-hydroxybutyric acid in each complex was determined by HPLC. The detection results were consistent with the structure of the complex. The specific data are shown in Table 2-1 below. The results of potassium content and 3-hydroxybutyric acid content of complex I prepared in Examples 2-6, 8-12 and 14-18 are consistent with those in Examples 1, 7 and 13 respectively; the results of calcium content and 3-hydroxybutyric acid content of complex II prepared in Examples 20-24, 26-30 and 32-36 are consistent with those in Examples 19, 25 and 31, respectively; and the results of magnesium content and 3-hydroxybutyric acid content of complex III prepared in Examples 38-42, 44-48 and 50-54 are consistent with those in Examples 37, 43 and 49, respectively. The sodium content in the complex IV of Examples 55, 61 and 67 was measured; and the content of 3-hydroxypentanoic acid in the complex was determined by HPLC. The detection results were consistent with the structure of the complex. The specific data are shown in Table 2-2 below. The results of sodium content and 3-hydroxyvaleric acid content of complex IV prepared in Examples 56-60, 62-66 and 68-72 are consistent with those in Examples 55, 61 and 67, respectively.

TABLE 2-1

| | Theoretical | Measured | Theoretical | Measured |
|---|---|---|---|---|
| | content | content | content | content |
| | (based on 3- | (based on 3- | (based | (based |
| | hydroxybutyric | hydroxybutyric | on metal | on metal |
| Example | acid) | acid) | ion) | ion) |
| 1 | 84.13% | 85.25% | 15.87% | 14.75% |
| 7 | 84.13% | 85.52% | 15.87% | 14.48% |
| 13 | 84.13% | 85.08% | 15.87% | 14.92% |
| 19 | 91.20% | 91.70% | 8.80% | 8.30% |
| 25 | 91.20% | 91.86% | 8.80% | 8.14% |
| 31 | 91.20% | 92.02% | 8.80% | 7.98% |
| 37 | 94.46% | 94.68% | 5.54% | 5.32% |
| 43 | 94.46% | 94.59% | 5.54% | 5.41% |
| 49 | 94.46% | 94.72% | 5.54% | 5.28% |

TABLE 2-2

| | | | Theoret- | |
|---|---|---|---|---|
| | Theoretical | Measured | ical | Measured |
| | content | content | content | content |
| | (based on 3- | (based on 3- | (based | (based |
| | hydroxypentanoic | hydroxypentanoic | on metal | on metal |
| Example | acid) | acid) | ion) | ion) |
| 55 | 91.10% | 91.18% | 8.90% | 8.82% |
| 61 | 91.10% | 91.24% | 8.90% | 8.76% |
| 67 | 91.10% | 91.05% | 8.90% | 8.95% |

### Example 77. Elemental Analysis

Elemental analysis was performed on the Complex I of Example 1: C 39.1%, H 6.1%; the elemental analysis results of Examples 2-18 are consistent with those of Example 1; and the results are consistent with the structure of 3-hydroxybutyric acid·potassium 3-hydroxybutyrate complex. Elemental analysis was performed on the Complex II of Example 19: C 42.2%, H 6.6%; the elemental analysis results of Examples 20-36 are consistent with those of Example 19; and the results are consistent with the structure of 3-hydroxybutyric acid·calcium 3-hydroxybutyrate complex. Elemental analysis was performed on the Complex III of Example 37: C 43.8%, H 6.9%; the elemental analysis results of Examples 38-54 are consistent with those of Example 37; and the results are consistent with the structure of 3-hydroxybutyric acid·magnesium 3-hydroxybutyrate complex. Elemental analysis was performed on the Complex IV of Example 55: C 46.5%, H 7.3%; the elemental analysis results of Examples 56-72 are consistent with those of Example 55; and the results are consistent with the structure of 3-hydroxypentanoic acid·sodium 3-hydroxypentanoate complex.

### Example 78. Raman Spectroscopy

The characteristic Raman spectrum of the complex can be obtained by Raman spectroscopy. FIG. 3 shows a Raman spectrogram of the Complex I of Example 1. The Complex I of Example 1 comprises the following characteristic absorption peaks at 3358.49 cm⁻¹, 2974.54 cm⁻¹, 2962.49 cm⁻¹, 2922.50 cm⁻¹, 2888.89 cm⁻¹, 2712.38 cm⁻¹, 1451.91 cm⁻¹, 1312.00 cm⁻¹, 1067.72 cm⁻¹, 912.01 cm⁻¹, 856.19 cm⁻¹, 752.02 cm⁻¹, and the error margin is +2 cm 1. The Raman spectra of the complexes prepared in Examples 2-18 are substantially the same as those in Example 1.

### Example 79. Thermogravimetric Analysis (TGA)

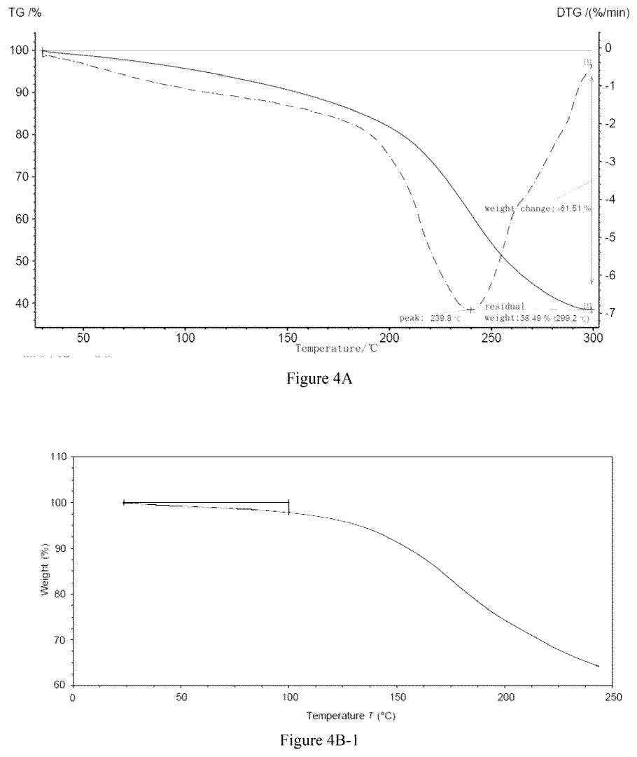

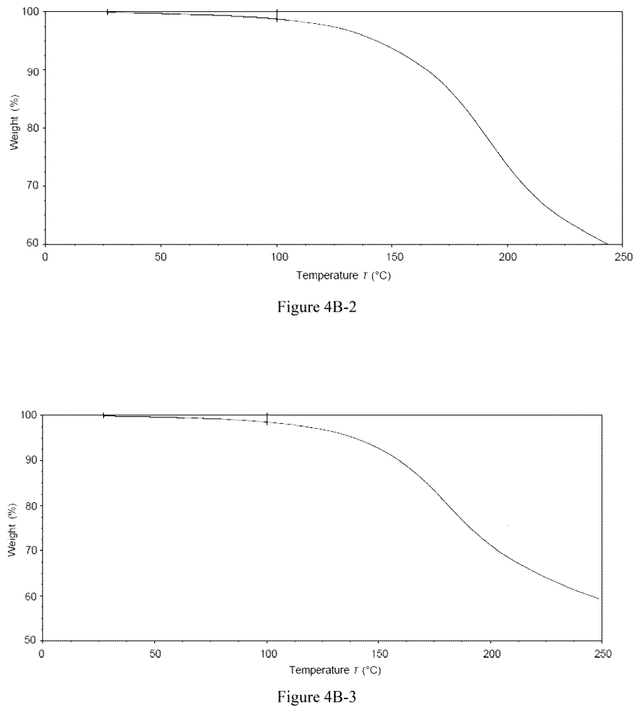

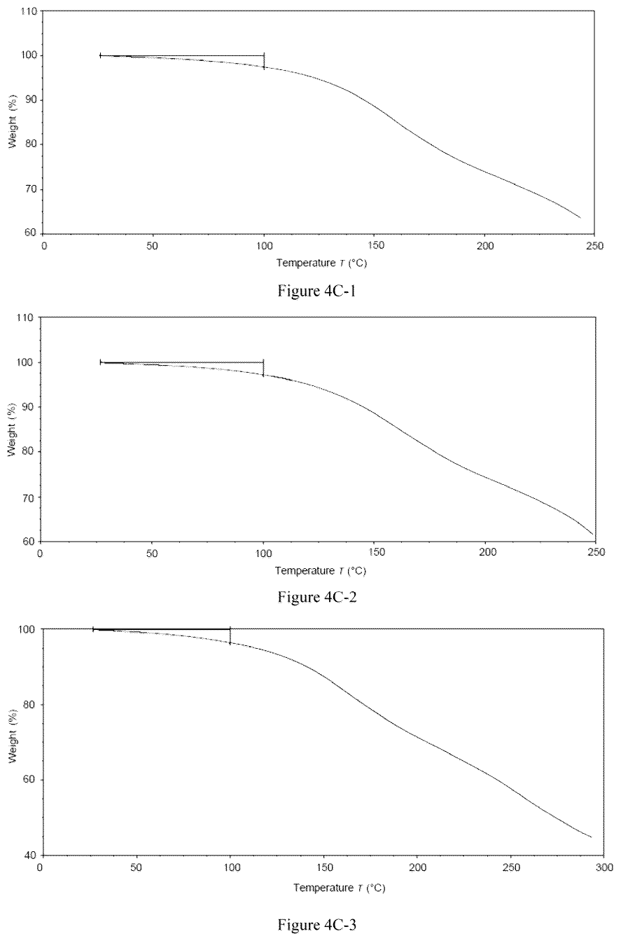

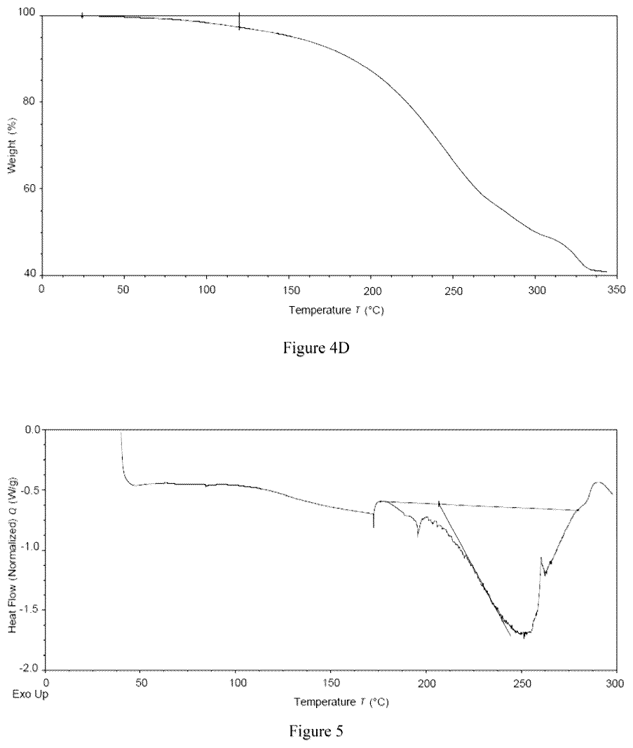

FIG. 4A shows a TGA diagram of the Complex I of Example 1. The complex has a weight loss of 61.51% when heated from 30° C. to 299° C. The TGA results of the complexes prepared in Examples 2-18 are substantially the same as those in Example 1. FIG. 4B-1 shows a TGA diagram of the Complex II of Example 19. The complex has a weight loss of 2.18% when heated from 23.5° C. to 100.0° C. The TGA results of the complexes prepared in Examples 20-24 are substantially the same as those in Example 19. FIG. 4B-2 shows a TGA diagram of the Complex II of Example 25. The complex has a weight loss of 1.22% when heated from 26.8° C. to 100.0° C. The TGA results of the complexes prepared in Examples 26-30 are substantially the same as those in Example 25. FIG. 4B-3 shows a TGA diagram of the Complex II of Example 31. The complex has a weight loss of 1.47% when heated from 27.2° C. to 100.0° C. The TGA results of the complexes prepared in Examples 32-36 are substantially the same as those in Example 31. FIG. 4C-1 shows a TGA diagram of the Complex III of Example 37. The complex has a weight loss of 2.47% when heated from 26.2° C. to 100.0° C. The TGA results of the complexes prepared in Examples 38-42 are substantially the same as those in Example 37. FIG. 4C-2 shows a TGA diagram of the Complex III of Example 43. The complex has a weight loss of 2.78% when heated from 26.9° C. to 100.0° C. The TGA results of the complexes prepared in Examples 44-48 are substantially the same as those in Example 43. FIG. 4C-3 shows a TGA diagram of the Complex III of Example 49. The complex has a weight loss of 3.59% when heated from 26.6° C. to 100.0° C. The TGA results of the complexes prepared in Examples 50-54 are substantially the same as those in Example 49. FIG. 4D shows a TGA diagram of the Complex IV of Example 55. The complex has a weight loss of 2.74% when heated from 24.2° C. to 120.0° C. The TGA results of the complexes prepared in Examples 56-72 are substantially the same as those in Example 55.

### Example 80. Differential Scanning Calorimetry (DSC)

FIG. 5 shows a DSC thermogram of the Complex I of Example 1, which contains an endothermic peak of 251.24° C.±3° C. The DSC results of the complexes prepared in Examples 2-18 are substantially the same as those in Example 1.

### Determination of Properties of Complexes of the Present Invention

### Example 81. Determination of Moisture Contents of Complexes of the Present Invention

For R-3-hydroxybutyric acid, potassium R-3-hydroxybutyrate, mixture of R-3-hydroxybutyric acid and potassium R-3-hydroxybutyrate, and R-3-hydroxybutyric acid·potassium R-3-hydroxybutyrate complex of Example 1, under a certain condition, KF moisture meter was used to determine the moisture contents at different time points. The experimental results are shown in Table 3A-1.

TABLE 3A-1

| | | moisture content (%) | | | | | | | |
|---|---|---|---|---|---|---|---|---|---|
| Condition | Sample | 30 min | 1 h | 2 h | 4 h | 6 h | 7 h | 11 h | 22 h |
| temperature | R-3-hydroxybutyric acid | 1.13 | 2.25 | 3.96 | 6.46 | 9.52 | 10.56 | 17.22 | 33.34 |
| 23° C. | Potassium R-3- | 1.04 | 1.91 | 3.19 | 4.93 | 7.65 | 8.24 | 16.72 | 32.68 |
| humidity | hydroxybutyrate | | | | | | | | |
| 92.5% | Mixture of R-3- | 1.09 | 2.09 | 3.32 | 5.43 | 7.86 | 8.35 | 16.88 | 32.48 |
| | hydroxybutyric acid and | | | | | | | | |
| | potassium R-3- | | | | | | | | |
| | hydroxybutyrate | | | | | | | | |
| | R-3-hydroxybutyric | 0.64 | 1.24 | 1.89 | 3.66 | 5.89 | 6.78 | 9.77 | 19.45 |
| | acid · potassium R-3- | | | | | | | | |
| | hydroxybutyrate complex | | | | | | | | |

For 3-hydroxybutyric acid, potassium 3-hydroxybutyrate, mixture of 3-hydroxybutyric acid and potassium 3-hydroxybutyrate, and 3-hydroxybutyric acid·potassium 3-hydroxybutyrate complex of Example 7, under a certain condition, KF moisture meter was used to determine the moisture contents at different time points. The experimental results are shown in Table 3A-2.

TABLE 3A-2

| | | moisture content (%) | | | | | | | |
|---|---|---|---|---|---|---|---|---|---|
| Condition | Sample | 30 min | 1 h | 2 h | 4 h | 6 h | 7 h | 11 h | 22 h |
| temperature | 3-hydroxybutyric acid | 1.32 | 2.37 | 4.15 | 6.59 | 9.68 | 10.71 | 17.33 | 33.51 |
| 23° C. | Potassium 3- | 1.14 | 1.93 | 3.22 | 4.98 | 7.77 | 8.34 | 16.82 | 32.86 |
| humidity | hydroxybutyrate | | | | | | | | |
| 92.5% | Mixture of 3- | 1.12 | 2.08 | 3.29 | 5.48 | 7.88 | 8.45 | 16.86 | 32.54 |
| | hydroxybutyric acid and | | | | | | | | |
| | potassium 3- | | | | | | | | |
| | hydroxybutyrate | | | | | | | | |
| | 3-hydroxybutyric | 0.68 | 1.28 | 1.93 | 3.68 | 5.92 | 6.84 | 9.67 | 19.25 |
| | acid · potassium 3- | | | | | | | | |
| | hydroxybutyrate complex | | | | | | | | |

For S-3-hydroxybutyric acid, potassium S-3-hydroxybutyrate, mixture of S-3-hydroxybutyric acid and potassium S-3-hydroxybutyrate, and S-3-hydroxybutyric acid·potassium S-3-hydroxybutyrate complex of Example 13, under a certain condition, KF moisture meter was used to determine the moisture contents at different time points. The experimental results are shown in Table 3A-3.

TABLE 3A-3

| | | moisture content (%) | | | | | | | |
|---|---|---|---|---|---|---|---|---|---|
| Condition | Sample | 30 min | 1 h | 2 h | 4 h | 6 h | 7 h | 11 h | 22 h |
| temperature | S-3-hydroxybutyric acid | 1.23 | 2.28 | 4.05 | 6.32 | 9.53 | 10.62 | 17.08 | 32.95 |
| 23° C. | Potassium S-3- | 1.06 | 1.92 | 3.22 | 4.97 | 7.55 | 8.34 | 16.82 | 32.75 |
| humidity | hydroxybutyrate | | | | | | | | |
| 92.5% | Mixture of S-3-hydroxybutyric | 1.12 | 2.19 | 3.22 | 5.47 | 7.82 | 8.33 | 16.54 | 32.65 |
| | acid and potassium S-3- | | | | | | | | |
| | hydroxybutyrate | | | | | | | | |
| | S-3-hydroxybutyric | 0.72 | 1.34 | 196 | 3.72 | 5.99 | 6.82 | 9.83 | 18.28 |
| | acid · potassium S-3- | | | | | | | | |
| | hydroxybutyrate complex | | | | | | | | |

For R-3-hydroxybutyric acid, calcium R-3-hydroxybutyrate, mixture of R-3-hydroxybutyric acid and calcium R-3-hydroxybutyrate, and R-3-hydroxybutyric acid·calcium R-3-hydroxybutyrate complex of Example 19, under a certain condition, KF moisture meter was used to determine the moisture contents at different time points. The experimental results are shown in Table 3B-1.

TABLE 3B-1

| | | moisture content (%) | | | | | | | |
|---|---|---|---|---|---|---|---|---|---|
| Condition | Sample | 30 min | 1 h | 2 h | 4 h | 6 h | 7 h | 11 h | 22 h |
| temperature | R-3-hydroxybutyric acid | 1.13 | 2.25 | 3.96 | 6.46 | 9.52 | 10.56 | 17.22 | 33.34 |
| 23° C. | Calcium R-3-hydroxybutyrate | 0.92 | 1.82 | 2.43 | 4.22 | 5.35 | 6.04 | 10.44 | 17.68 |
| humidity | Mixture of R-3- | 1.09 | 2.19 | 3.09 | 5.43 | 6.96 | 7.44 | 10.88 | 19.85 |
| 92.5% | hydroxybutyric acid and | | | | | | | | |
| | calcium R-3-hydroxybutyrate | | | | | | | | |
| | R-3-hydroxybutyric | 0.54 | 1.02 | 1.96 | 3.26 | 4.79 | 5.39 | 8.65 | 14.45 |
| | acid · calcium R-3- | | | | | | | | |
| | hydroxybutyrate complex | | | | | | | | |

For 3-hydroxybutyric acid, calcium 3-hydroxybutyrate, mixture of 3-hydroxybutyric acid and calcium 3-hydroxybutyrate, and 3-hydroxybutyric acid·calcium 3-hydroxybutyrate complex of Example 25, under a certain condition, KF moisture meter was used to determine the moisture contents at different time points. The experimental results are shown in Table 3B-2.

TABLE 3B-2

| | | moisture content (%) | | | | | | | |
|---|---|---|---|---|---|---|---|---|---|
| Condition | Sample | 30 min | 1 h | 2 h | 4 h | 6 h | 7 h | 11 h | 22 h |
| temperature | 3-hydroxybutyric acid | 1.32 | 2.37 | 4.15 | 6.59 | 9.68 | 10.71 | 17.33 | 33.51 |
| 23° C. | Calcium 3-hydroxybutyrate | 0.97 | 1.81 | 2.34 | 4.12 | 5.22 | 6.01 | 10.24 | 17.72 |
| humidity | Mixture of 3- | 1.19 | 2.09 | 3.21 | 5.34 | 6.87 | 7.64 | 10.72 | 19.96 |
| 92.5% | hydroxybutyric acid and | | | | | | | | |
| | calcium 3-hydroxybutyrate | | | | | | | | |
| | 3-hydroxybutyric | 0.56 | 1.12 | 1.86 | 3.19 | 4.68 | 5.45 | 8.72 | 14.43 |
| | acid · calcium 3- | | | | | | | | |
| | hydroxybutyrate complex | | | | | | | | |

For S-3-hydroxybutyric acid, calcium S-3-hydroxybutyrate, mixture of S-3-hydroxybutyric acid and calcium S-3-hydroxybutyrate, and S-3-hydroxybutyric acid·calcium S-3-hydroxybutyrate complex of Example 31, under a certain condition, KF moisture meter was used to determine the moisture contents at different time points. The experimental results are shown in Table 3B-3.

TABLE 3B-3

| | | moisture content (%) | | | | | | | |
|---|---|---|---|---|---|---|---|---|---|
| Condition | Sample | 30 min | 1 h | 2 h | 4 h | 6 h | 7 h | 11 h | 22 h |
| temperature | S-3-hydroxybutyric acid | 1.23 | 2.28 | 4.05 | 6.32 | 9.53 | 10.62 | 17.08 | 32.95 |
| 23° C. | Calcium S-3-hydroxybutyrate | 0.88 | 1.92 | 2.25 | 4.18 | 5.24 | 6.25 | 10.34 | 17.55 |
| humidity | Mixture of S-3-hydroxybutyric | 1.13 | 2.06 | 3.12 | 5.27 | 6.83 | 7.26 | 10.75 | 19.66 |
| 92.5% | acid and calcium S-3- | | | | | | | | |
| | hydroxybutyrate | | | | | | | | |
| | S-3-hydroxybutyric | 0.52 | 0.98 | 1.88 | 3.05 | 4.47 | 5.22 | 8.41 | 13.98 |
| | acid · calcium S-3- | | | | | | | | |
| | hydroxybutyrate complex | | | | | | | | |

For R-3-hydroxybutyric acid, magnesium R-3-hydroxybutyrate, mixture of R-3-hydroxybutyric acid and magnesium R-3-hydroxybutyrate, and R-3-hydroxybutyric acid·magnesium R-3-hydroxybutyrate complex of Example 37, under a certain condition, KF moisture meter was used to determine the moisture contents at different time points. The experimental results are shown in Table 3C-1.

TABLE 3C-1

| | | moisture content (%) | | | | | | | |
|---|---|---|---|---|---|---|---|---|---|
| Condition | Sample | 30 min | 1 h | 2 h | 4 h | 6 h | 7 h | 11 h | 22 h |
| temperature | R-3-hydroxybutyric acid | 1.13 | 2.25 | 3.96 | 6.46 | 9.52 | 10.56 | 17.22 | 33.34 |
| 23° C. | Magnesium R-3- | 0.82 | 1.22 | 2.13 | 4.22 | 6.35 | 8.04 | 12.44 | 18.68 |
| humidity | hydroxybutyrate | | | | | | | | |
| 92.5% | Mixture of R-3- | 0.89 | 2.16 | 3.02 | 5.27 | 7.83 | 9.26 | 13.55 | 19.46 |
| | hydroxybutyric acid and | | | | | | | | |
| | magnesium R-3- | | | | | | | | |
| | hydroxybutyrate | | | | | | | | |
| | R-3-hydroxybutyric | 0.57 | 0.92 | 1.76 | 3.16 | 4.59 | 5.69 | 8.65 | 14.45 |
| | acid · magnesium R-3- | | | | | | | | |
| | hydroxybutyrate complex | | | | | | | | |

For 3-hydroxybutyric acid, magnesium 3-hydroxybutyrate, mixture of 3-hydroxybutyric acid and magnesium 3-hydroxybutyrate, and 3-hydroxybutyric acid·magnesium 3-hydroxy butyrate complex of Example 43, under a certain condition, KF moisture meter was used to determine the moisture contents at different time points. The experimental results are shown in Table 3C-2.

TABLE 3C-2

| | | moisture content (%) | | | | | | | |
|---|---|---|---|---|---|---|---|---|---|
| Condition | Sample | 30 min | 1 h | 2 h | 4 h | 6 h | 7 h | 11 h | 22 h |
| temperature | 3-hydroxybutyric acid | 1.32 | 2.37 | 4.15 | 6.59 | 9.68 | 10.71 | 17.33 | 33.51 |
| 23° C. | Magnesium 3- | 0.81 | 1.25 | 2.23 | 4.12 | 6.43 | 8.14 | 12.42 | 18.66 |
| humidity | hydroxybutyrate | | | | | | | | |
| 92.5% | Mixture of 3- | 0.92 | 2.22 | 3.07 | 5.33 | 7.68 | 9.13 | 13.57 | 19.42 |
| | hydroxybutyric acid and | | | | | | | | |
| | magnesium 3- | | | | | | | | |
| | hydroxybutyrate | | | | | | | | |
| | 3-hydroxybutyric | 0.56 | 0.97 | 1.82 | 3.26 | 4.62 | 5.57 | 8.75 | 14.68 |
| | acid · magnesium 3- | | | | | | | | |
| | hydroxybutyrate complex | | | | | | | | |

For S-3-hydroxybutyric acid, magnesium S-3-hydroxybutyrate, mixture of S-3-hydroxybutyric acid and magnesium S-3-hydroxybutyrate, and S-3-hydroxybutyric acid·magnesium S-3-hydroxybutyrate complex of Example 49, under a certain condition, KF moisture meter was used to determine the moisture contents at different time points. The experimental results are shown in Table 3C-3.

TABLE 3C-3

| | | moisture content (%) | | | | | | | |
|---|---|---|---|---|---|---|---|---|---|
| Condition | Sample | 30 min | 1 h | 2 h | 4 h | 6 h | 7 h | 11 h | 22 h |
| temperature | S-3-hydroxybutyric acid | 1.23 | 2.28 | 4.05 | 6.32 | 9.53 | 10.62 | 17.08 | 32.95 |
| 23° C. | Magnesium S-3- | 0.88 | 1.28 | 2.43 | 4.22 | 6.37 | 8.24 | 12.35 | 18.54 |
| humidity | hydroxybutyrate | | | | | | | | |
| 92.5% | Mixture of S-3-hydroxybutyric | 0.95 | 2.16 | 3.17 | 5.24 | 7.75 | 9.24 | 13.37 | 19.57 |
| | acid and magnesium S-3- | | | | | | | | |
| | hydroxybutyrate | | | | | | | | |
| | S-3-hydroxybutyric | 0.58 | 0.98 | 1.79 | 3.16 | 4.58 | 5.64 | 8.86 | 14.74 |
| | acid · magnesium S-3- | | | | | | | | |
| | hydroxybutyrate complex | | | | | | | | |

For R-3-hydroxypentanoic acid, sodium R-3-hydroxypentanoate, mixture of R-3-hydroxypentanoic acid and sodium R-3-hydroxypentanoate, and R-3-hydroxypentanoic acid·sodium R-3-hydroxypentanoate complex of Example 55, under a certain condition, KF moisture meter was used to determine the moisture contents at different time points. The experimental results are shown in Table 3D-1.

TABLE 3D-1

| | | moisture content (%) | | | | | | | |
|---|---|---|---|---|---|---|---|---|---|
| Condition | Sample | 30 min | 1 h | 2 h | 4 h | 6 h | 7 h | 11 h | 22 h |
| temperature | R-3-hydroxypentanoic acid | 0.94 | 1.88 | 3.30 | 5.38 | 7.93 | 8.80 | 14.35 | 27.78 |
| 23° C. | Sodium R-3- | 0.87 | 1.68 | 2.83 | 4.36 | 6.13 | 6.70 | 10.57 | 20.57 |
| humidity | hydroxypentanoate | | | | | | | | |
| 92.5% | Mixture of R-3- | 1.08 | 1.99 | 3.16 | 4.53 | 5.80 | 6.20 | 9.07 | 16.54 |
| | hydroxypentanoic acid and | | | | | | | | |
| | sodium R-3-hydroxypentanoate | | | | | | | | |
| | R-3-hydroxypentanoic | 0.45 | 0.85 | 1.63 | 2.72 | 4.08 | 4.49 | 7.21 | 14.54 |
| | acid · sodium R-3- | | | | | | | | |
| | hydroxypentanoate complex | | | | | | | | |

For 3-hydroxypentanoic acid, sodium 3-hydroxypentanoate, mixture of 3-hydroxypentanoic acid and sodium 3-hydroxypentanoate, and 3-hydroxypentanoic acid·sodium 3-hydroxypentanoate complex of Example 61, under a certain condition, KF moisture meter was used to determine the moisture contents at different time points. The experimental results are shown in Table 3D-2.

TABLE 3D-2

| | | moisture content (%) | | | | | | | |
|---|---|---|---|---|---|---|---|---|---|
| Condition | Sample | 30 min | 1 h | 2 h | 4 h | 6 h | 7 h | 11 h | 22 h |
| temperature | 3-hydroxypentanoic acid | 1.10 | 1.98 | 3.46 | 5.49 | 8.07 | 8.93 | 14.44 | 27.93 |
| 23° C. | Sodium 3- | 0.96 | 1.83 | 3.02 | 4.46 | 6.28 | 6.96 | 10.72 | 20.79 |
| humidity | hydroxypentanoate | | | | | | | | |
| 92.5% | Mixture of 3- | 1.19 | 2.05 | 3.30 | 4.68 | 5.99 | 6.52 | 9.48 | 16.94 |
| | hydroxypentanoic acid | | | | | | | | |
| | and sodium 3- | | | | | | | | |
| | hydroxypentanoate | | | | | | | | |
| | 3-hydroxypentanoic | 0.51 | 0.98 | 1.71 | 2.82 | 4.16 | 4.60 | 7.28 | 14.69 |
| | acid · sodium 3- | | | | | | | | |
| | hydroxypentanoate | | | | | | | | |
| | complex | | | | | | | | |

For S-3-hydroxypentanoic acid, sodium S-3-hydroxypentanoate, mixture of S-3-hydroxypentanoic acid and sodium S-3-hydroxypentanoate, and S-3-hydroxypentanoic acid·sodium S-3-hydroxypentanoate complex of Example 67, under a certain condition, KF moisture meter was used to determine the moisture contents at different time points. The experimental results are shown in Table 3D-3.

TABLE 3D-3

| | | moisture content (%) | | | | | | | |
|---|---|---|---|---|---|---|---|---|---|
| Condition | Sample | 30 min | 1 h | 2 h | 4 h | 6 1 | 7 h | 11 h | 22 h |
| temperature | S-3-hydroxypentanoic acid | 1.03 | 1.90 | 3.38 | 5.27 | 7.94 | 8.85 | 14.23 | 27.46 |
| 23° C. | Sodium S-3- | 0.91 | 1.73 | 2.91 | 4.43 | 6.22 | 6.79 | 10.61 | 20.44 |
| humidity | hydroxypentanoate | | | | | | | | |
| 92.5% | Mixture of S-3- | 1.18 | 2.03 | 3.32 | 4.89 | 6.03 | 6.37 | 9.90 | 15.79 |
| | hydroxypentanoic acid and | | | | | | | | |
| | sodium S-3-hydroxypentanoate | | | | | | | | |
| | S-3-hydroxypentanoic | 0.59 | 1.15 | 1.54 | 2.73 | 3.99 | 4.43 | 7.03 | 14.11 |
| | acid · sodium S-3- | | | | | | | | |
| | hydroxypentanoate complex | | | | | | | | |

The above results show that the moisture content of the Complexes I to IV of the invention is significantly lower than that of the corresponding acid, salt, acid and salt mixture, which can increase the application scenarios of complex products. Because solids of acid have strong hygroscopicity and are easy to deliquesce, they cannot be well used in the field of solid preparations, which greatly limits their application in the fields of solid nutrition and dietary supplements. The hygroscopicity of the Complexes I to IV of the invention is obviously superior to that of the corresponding acid, salt, acid and salt mixture. In addition, simple mixture of acid and salt is prone to uneven mixing. Therefore, the application range of complex is wider, especially suitable for the preparation and application of solid preparations.

### Example 82. Stability of Complexes of the Present Invention

For R-3-hydroxybutyric acid, potassium R-3-hydroxybutyrate, mixture of R-3-hydroxybutyric acid and potassium R-3-hydroxybutyrate, and R-3-hydroxybutyric acid·potassium R-3-hydroxybutyrate complex of Example 1, the stability was measured at 30° C., 40° C., 60° C., 70° C. and 80° C. The experimental results are shown in Table 4A-1.

TABLE 4A-1

| | Stability | | | | | |
|---|---|---|---|---|---|---|
| Sample | 30° C. | 40° C. | 50° C. | 60° C. | 70° C. | 80° C. |
| R-3-hydroxybutyric | unchanged | partially | melted | melted | melted | melted |
| acid | | melted | | | | |
| Potassium R-3- | unchanged | partially | melted | melted | melted | melted |
| hydroxybutyrate | | melted | | | | |
| Mixture of R-3- | unchanged | unchanged | partially | melted | melted | melted |
| hydroxybutyric acid | | | melted | | | |
| and potassium R-3- | | | | | | |
| hydroxybutyrate | | | | | | |
| R-3-hydroxybutyric | unchanged | unchanged | unchanged | unchanged | partially | melted |
| acid · potassium R-3- | | | | | melted | |
| hydroxybutyrate | | | | | | |
| complex | | | | | | |

For 3-hydroxybutyric acid, potassium 3-hydroxybutyrate, mixture of 3-hydroxybutyric acid and potassium 3-hydroxybutyrate, and 3-hydroxybutyric acid·potassium 3-hydroxybutyrate complex of Example 7, the stability was measured at 30° C., 40° C., 60° C., 70° C. and 80° C. The experimental results are shown in Table 4A-2.

TABLE 4A-2

| | Stability | | | | | |
|---|---|---|---|---|---|---|
| Sample | 30° C. | 40° C. | 50° C. | 60° C. | 70° C. | 80° C. |
| 3-hydroxybutyric | unchanged | partially | Melted | melted | melted | melted |
| acid | | melted | | | | |
| Potassium 3- | unchanged | partially | Melted | melted | melted | melted |
| hydroxybutyrate | | melted | | | | |
| Mixture of 3- | unchanged | unchanged | partially | melted | melted | melted |
| hydroxybutyric acid | | | melted | | | |
| and potassium 3- | | | | | | |
| hydroxybutyrate | | | | | | |
| 3-hydroxybutyric | unchanged | unchanged | Unchanged | unchanged | partially | melted |
| acid · potassium 3- | | | | | melted | |
| hydroxybutyrate | | | | | | |
| complex | | | | | | |

For S-3-hydroxybutyric acid, potassium S-3-hydroxybutyrate, mixture of S-3-hydroxybutyric acid and potassium S-3-hydroxybutyrate, and S-3-hydroxybutyric acid·potassium S-3-hydroxybutyrate complex of Example 13, the stability was measured at 30° C., 40° C., 60° C., 70° C. and 80° C. The experimental results are shown in Table 4A-3.

TABLE 4A-3

| | Stability | | | | | |
|---|---|---|---|---|---|---|
| Sample | 30° C. | 40° C. | 50° C. | 60° C. | 70° C. | 80° C. |
| S-3-hydroxybutyric | unchanged | partially | melted | melted | melted | melted |
| acid | | melted | | | | |
| Potassium S-3- | unchanged | partially | melted | melted | melted | melted |
| hydroxybutyrate | | melted | | | | |
| Mixture of S-3- | unchanged | unchanged | partially | melted | melted | melted |
| hydroxybutyric acid | | | melted | | | |
| and potassium S-3- | | | | | | |
| hydroxybutyrate | | | | | | |
| S-3-hydroxybutyric | unchanged | unchanged | unchanged | unchanged | partially | melted |
| acid · potassium S-3- | | | | | melted | |
| hydroxybutyrate | | | | | | |
| complex | | | | | | |

For R-3-hydroxybutyric acid, calcium R-3-hydroxybutyrate, mixture of R-3-hydroxybutyric acid and calcium R-3-hydroxybutyrate, and R-3-hydroxybutyric acid·calcium R-3-hydroxybutyrate complex of Example 19, the stability was measured at 30° C., 40° C., 60° C., 70° C. and 80° C. The experimental results are shown in Table 4B-1.

TABLE 4B-1

| | Stability | | | | | |
|---|---|---|---|---|---|---|
| Sample | 30° C. | 40° C. | 50° C. | 60° C. | 70° C. | 80° C. |
| R-3-hydroxybutyric | unchanged | partially | melted | melted | melted | melted |
| acid | | melted | | | | |
| Calcium R-3- | unchanged | unchanged | partially | melted | melted | melted |
| hydroxybutyrate | | | melted | | | |
| Mixture of R-3- | unchanged | unchanged | partially | melted | melted | melted |
| hydroxybutyric acid | | | melted | | | |
| and calcium R-3- | | | | | | |
| hydroxybutyrate | | | | | | |
| R-3-hydroxybutyric | unchanged | unchanged | unchanged | unchanged | partially | melted |
| acid · calcium R-3- | | | | | melted | |
| hydroxybutyrate | | | | | | |
| complex | | | | | | |

For 3-hydroxybutyric acid, calcium 3-hydroxybutyrate, mixture of 3-hydroxybutyric acid and calcium 3-hydroxybutyrate, and 3-hydroxybutyric acid·calcium 3-hydroxybutyrate complex of Example 25, the stability was measured at 30° C., 40° C., 60° C., 70° C. and 80° C. The experimental results are shown in Table 4B-2.

TABLE 4B-2

| | Stability | | | | | |
|---|---|---|---|---|---|---|
| Sample | 30° C. | 40° C. | 50° C. | 60° C. | 70° C. | 80° C. |
| 3-hydroxybutyric | unchanged | partially | melted | melted | melted | melted |
| acid | | melted | | | | |
| Calcium 3- | unchanged | unchanged | partially | melted | melted | melted |
| hydroxybutyrate | | | melted | | | |
| Mixture of 3- | unchanged | unchanged | partially | melted | melted | melted |
| hydroxybutyric acid | | | melted | | | |
| and calcium 3- | | | | | | |
| hydroxybutyrate | | | | | | |
| 3-hydroxybutyric | unchanged | unchanged | unchanged | unchanged | partially | melted |
| acid · calcium 3- | | | | | melted | |
| hydroxybutyrate | | | | | | |
| complex | | | | | | |

For S-3-hydroxybutyric acid, calcium S-3-hydroxybutyrate, mixture of S-3-hydroxybutyric acid and calcium S-3-hydroxybutyrate, and S-3-hydroxybutyric acid·calcium S-3-hydroxybutyrate complex of Example 31, the stability was measured at 30° C., 40° C., 60° C., 70° C. and 80° C. The experimental results are shown in Table 4B-3.

TABLE 4B-3

| | Stability | | | | | |
|---|---|---|---|---|---|---|
| Sample | 30° C. | 40° C. | 50° C. | 60° C. | 70° C. | 80° C. |
| S-3-hydroxybutyric | unchanged | partially | melted | melted | melted | melted |
| acid | | melted | | | | |
| Calcium S-3- | unchanged | unchanged | partially | melted | melted | melted |
| hydroxybutyrate | | | melted | | | |
| Mixture of S-3- | unchanged | unchanged | partially | melted | melted | melted |
| hydroxybutyric acid | | | melted | | | |
| and calcium S-3- | | | | | | |
| hydroxybutyrate | | | | | | |
| S-3-hydroxybutyric | unchanged | unchanged | unchanged | unchanged | partially | melted |
| acid · calcium S-3- | | | | | melted | |
| hydroxybutyrate | | | | | | |
| complex | | | | | | |

For R-3-hydroxybutyric acid, magnesium R-3-hydroxybutyrate, mixture of R-3-hydroxybutyric acid and magnesium R-3-hydroxybutyrate, and R-3-hydroxybutyric acid·magnesium R-3-hydroxybutyrate complex of Example 37, the stability was measured at 30° C., 40° C., 60° C., 70° C. and 80° C. The experimental results are shown in Table 4C-1.

TABLE 4C-1

| | Stability | | | | | |
|---|---|---|---|---|---|---|
| Sample | 30° C. | 40° C. | 50° C. | 60° C. | 70° C. | 80° C. |
| R-3-hydroxybutyric | unchanged | partially | melted | melted | melted | melted |
| acid | | melted | | | | |
| magnesium R-3- | unchanged | unchanged | partially | melted | melted | melted |
| hydroxybutyrate | | | melted | | | |
| Mixture of R-3- | unchanged | unchanged | partially | melted | melted | melted |
| hydroxybutyric acid | | | melted | | | |
| and magnesium R-3- | | | | | | |
| hydroxybutyrate | | | | | | |
| R-3-hydroxybutyric | unchanged | unchanged | unchanged | unchanged | partially | melted |
| acid · magnesium | | | | | melted | |
| R-3-hydroxybutyrate | | | | | | |
| complex | | | | | | |

For 3-hydroxybutyric acid, magnesium 3-hydroxybutyrate, mixture of 3-hydroxybutyric acid and magnesium 3-hydroxybutyrate, and 3-hydroxybutyric acid·magnesium 3-hydroxybutyrate complex of Example 43, the stability was measured at 30° C., 40° C., 60° C., 70° C. and 80° C. The experimental results are shown in Table 4C-2.

TABLE 4C-2

| | Stability | | | | | |
|---|---|---|---|---|---|---|
| Sample | 30° C. | 40° C. | 50° C. | 60° C. | 70° C. | 80° C. |
| 3-hydroxybutyric | unchanged | partially | melted | Melted | melted | melted |
| acid | | melted | | | | |
| Magnesium 3- | unchanged | unchanged | partially | Melted | melted | melted |
| hydroxybutyrate | | | melted | | | |
| Mixture of 3- | unchanged | unchanged | partially | Melted | melted | melted |
| hydroxybutyric acid | | | melted | | | |
| and magnesium 3- | | | | | | |
| hydroxybutyrate | | | | | | |
| 3-hydroxybutyric | unchanged | unchanged | unchanged | Unchanged | partially | melted |
| acid · magnesium | | | | | melted | |
| 3-hydroxybutyrate | | | | | | |
| complex | | | | | | |

For S-3-hydroxybutyric acid, magnesium S-3-hydroxybutyrate, mixture of S-3-hydroxybutyric acid and magnesium S-3-hydroxybutyrate, and S-3-hydroxybutyric acid·magnesium S-3-hydroxybutyrate complex of Example 49, the stability was measured at 30° C., 40° C., 60° C., 70° C. and 80° C. The experimental results are shown in Table 4C-3.

TABLE 4C-3

| | Stability | | | | | |
|---|---|---|---|---|---|---|
| Sample | 30° C. | 40° C. | 50° C. | 60° C. | 70° C. | 80° C. |
| S-3-hydroxybutyric | unchanged | partially | melted | melted | melted | melted |
| acid | | melted | | | | |
| Magnesium S-3- | unchanged | unchanged | partially | melted | melted | melted |
| hydroxybutyrate | | | melted | | | |
| Mixture of S-3- | unchanged | unchanged | partially | melted | melted | melted |
| hydroxybutyric acid | | | melted | | | |
| and magnesium S-3- | | | | | | |
| hydroxybutyrate | | | | | | |
| S-3-hydroxybutyric | unchanged | unchanged | unchanged | unchanged | partially | melted |
| acid · magnesium S- | | | | | melted | |
| 3-hydroxybutyrate | | | | | | |
| complex | | | | | | |

For R-3-hydroxypentanoic acid, sodium R-3-hydroxypentanoate, mixture of R-3-hydroxypentanoic acid and sodium R-3-hydroxypentanoate, and R-3-hydroxypentanoic acid·sodium R-3-hydroxypentanoate complex of Example 55, the stability was measured at 30° C., 40° C., 60° C., 70° C. and 80° C. The experimental results are shown in Table 4D-1.

TABLE 4D-1

| | Stability | | | | | |
|---|---|---|---|---|---|---|
| Sample | 30° C. | 40° C. | 50° C. | 60° C. | 70° C. | 80° C. |
| R-3-hydroxypentanoic | unchanged | partially | melted | melted | melted | melted |
| acid | | melted | | | | |
| Sodium R-3- | unchanged | unchanged | unchanged | unchanged | unchanged | unchanged |
| hydroxypentanoate | | | | | | |
| Mixture of R-3- | unchanged | partially | partially | partially | partially | melted |
| hydroxypentanoic acid | | melted | melted | melted | melted | |
| and sodium R-3- | | | | | | |
| hydroxypentanoate | | | | | | |
| R-3-hydroxypentanoic | unchanged | unchanged | unchanged | unchanged | unchanged | unchanged |
| acid · sodium R-3- | | | | | | |
| hydroxypentanoate | | | | | | |
| complex | | | | | | |

For 3-hydroxypentanoic acid, sodium 3-hydroxypentanoate, mixture of 3-hydroxypentanoic acid and sodium 3-hydroxypentanoate, and 3-hydroxypentanoic acid·sodium 3-hydroxypentanoate complex of Example 61, the stability was measured at 30° C., 40° C., 60° C., 70° C. and 80° C. The experimental results are shown in Table 4D-2.

TABLE 4D-2

| | Stability | | | | | |
|---|---|---|---|---|---|---|
| Sample | 30° C. | 40° C. | 50° C. | 60° C. | 70° C. | 80° C. |
| 3-hydroxypentanoic | unchanged | partially | melted | melted | melted | melted |
| acid | | melted | | | | |
| Sodium 3- | unchanged | unchanged | unchanged | unchanged | unchanged | unchanged |
| hydroxypentanoate | | | | | | |
| Mixture of 3- | unchanged | partially | partially | partially | partially | melted |
| hydroxypentanoic | | melted | melted | melted | melted | |
| acid and sodium 3- | | | | | | |
| hydroxypentanoate | | | | | | |
| 3-hydroxypentanoic | unchanged | unchanged | unchanged | unchanged | unchanged | unchanged |
| acid · sodium 3- | | | | | | |
| hydroxypentanoate | | | | | | |
| complex | | | | | | |

For S-3-hydroxypentanoic acid, sodium S-3-hydroxypentanoate, mixture of S-3-hydroxypentanoic acid and sodium S-3-hydroxypentanoate, and S-3-hydroxypentanoic acid·sodium S-3-hydroxypentanoate complex of Example 67, the stability was measured at 30° C., 40° C., 60° C., 70° C. and 80° C. The experimental results are shown in Table 4D-3.

TABLE 4D-3

| | Stability | | | | | |
|---|---|---|---|---|---|---|
| Sample | 30° C. | 40° C. | 50° C. | 60° C. | 70° C. | 80° C. |
| S-3- | unchanged | partially | melted | melted | melted | melted |
| hydroxypentanoic | | melted | | | | |
| acid | | | | | | |
| Sodium S-3- | unchanged | unchanged | unchanged | unchanged | unchanged | unchanged |
| hydroxypentanoate | | | | | | |
| Mixture of S-3- | unchanged | partially | partially | partially | partially | melted |
| hydroxypentanoic | | melted | melted | melted | melted | |
| acid and sodium S-3- | | | | | | |
| hydroxypentanoate | | | | | | |
| S-3- | unchanged | unchanged | unchanged | unchanged | unchanged | unchanged |
| hydroxypentanoic | | | | | | |
| acid · sodium S-3- | | | | | | |
| hydroxypentanoate | | | | | | |
| complex | | | | | | |

The above results show that the stability of Complexes I-IV of the present invention is very high, which is significantly superior to the corresponding acids and acid and salt mixtures.

The invention solves the problems of strong acidity, intestinal side effects, high hygroscopicity and poor stability of BHB acid and BHP acid, and simultaneously solves the electrolyte imbalance caused by high salt load of BHB salt and BHP salt. The complex shows better comprehensive effect than acid or salt alone or simple physically mixed components, and has appropriate hygroscopicity and stability, so that it is particularly suitable for the preparation of solid preparations. At a proper dosage, the complex of the invention has a good ketogenic effect. In addition, the complex prepared by the invention has high purity, uniform particle size distribution, good fluidity, low adhesiveness and good bioavailability; and the preparation process is controllable, at low cost, and environmentally friendly.

The above are only the preferred embodiments of the present invention, and are not intended to limit the present invention. Those skilled in the art can make many changes, modifications, substitutions and variations on these embodiments without departing from the principles and purposes of the invention, and the scope of the invention is defined by the claims and their equivalents.

1. A complex comprising 3-hydroxybutyric acid; and one or more of potassium 3-hydroxybutyrate, calcium 3-hydroxybutyrate, or magnesium 3-hydroxybutyrate.

2. The complex of claim 1, wherein anions in the complex structure include 3-hydroxybutyric acid anions; and cations include one or more of potassium ions, calcium ions and magnesium ions, as well as hydrogen ions.

3. The complex of claim 1 or 2, wherein the ratio of 3-hydroxybutyric acid to one or more of potassium 3-hydroxybutyrate, calcium 3-hydroxybutyrate or magnesium 3-hydroxybutyrate is 1:10 to 10:1.

4. The complex of any one of claims 1 to 3, wherein the complex contains not less than 50% of R configuration, not more than 50% of S configuration; or more than 50% of S configuration, less than 50% of R configuration.

5. The complex of any one of claims 1 to 4, wherein the complex is 3-hydroxybutyric acid·potassium 3-hydroxybutyrate or 3-hydroxybutyric acid·calcium 3-hydroxybutyrate or 3-hydroxybutyric acid·magnesium 3-hydroxybutyrate or mixture thereof.

6. The complex of any one of claims 1 to 5, wherein the complex has the following structure: 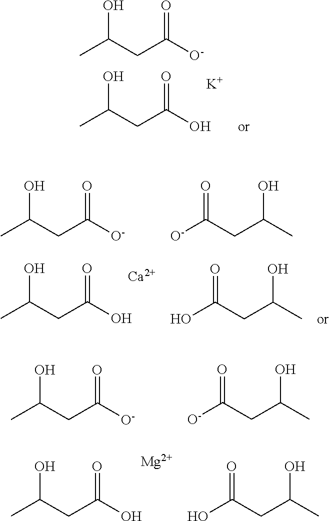
or mixture thereof.

7. A 3-hydroxybutyric acid·3-hydroxybutyrate salt complex, and the salt is a potassium salt, a calcium salt and/or a magnesium salt.

8. The complex of any one of claims 1 to 7, wherein the complex is a R-3-hydroxybutyric acid·R-3-hydroxybutyrate salt complex and/or a S-3-hydroxybutyric acid·S-3-hydroxybutyrate salt complex.

9. The complex of any one of claims 1 to 7, wherein the complex contains not less than 50% of R-3-hydroxybutyric acid·R-3-hydroxybutyrate salt, not more than 50% of S-3-hydroxybutyric acid·S-3-hydroxybutyrate salt; or more than 50% of S-3-hydroxybutyric acid·S-3-hydroxybutyrate salt, less than 50% of R-3-hydroxybutyric acid·R-3-hydroxybutyrate salt.

10. The complex of claim 8 or 9, wherein the complex has the following structure:

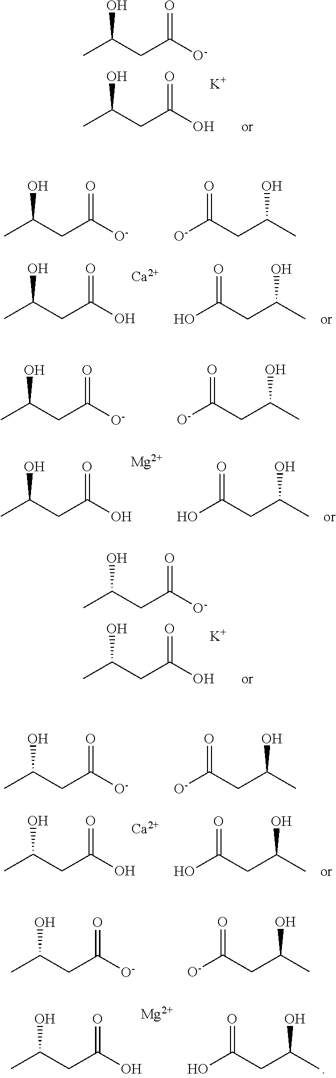

11. The complex of any one of claims 1 to 10, wherein the complex is in crystalline form.

12. The complex of any one of claims 1 to 11, wherein the X-ray powder diffraction pattern of the potassium salt complex comprises peaks at diffraction angles (2θ) of 6.7±0.2°, 19.6±0.2°, 24.9±0.2°, and 27.1±0.2°.

13. The complex of claim 12, wherein the X-ray powder diffraction pattern of the potassium salt complex further comprises one or more peaks at diffraction angles (2θ) of 13.4±0.2°, 21.4±0.2°, 26.0±0.2°, 32.5±0.2°.

14. The complex of claim 12 or 13, wherein the X-ray powder diffraction pattern of the potassium salt complex further comprises one or more peaks at diffraction angles (2θ) of 20.2±0.2°, 23.4±0.2°, 28.2±0.2°, 34.0±0.2°.

15. The complex of any one of claims 1 to 14, wherein the X-ray powder diffraction pattern of the potassium salt complex is shown in FIG. 1.

16. The complex of any one of claims 1 to 15, wherein the infrared spectrum of the potassium salt complex has the following absorption bands, expressed in reciprocal wavelengths (cm⁻¹) (±2 cm⁻¹): 2972, 2933, 1715, 1574, 1304, 1196, 1126, 1065, 951, 854.

17. The complex of any one of claims 1 to 11, wherein the infrared spectrum of the calcium salt complex has the following absorption bands, expressed in reciprocal wavelengths (cm⁻¹) (±2 cm⁻¹): 2974, 2936, 1715, 1558, 1506, 1300, 1196, 1126, 1065, 951, 854.

18. The complex of any one of claims 1 to 11, wherein the infrared spectrum of the magnesium salt complex has the following absorption bands, expressed in reciprocal wavelengths (cm⁻¹) (±2 cm⁻¹): 2976, 2936, 1713, 1321, 1207, 1088, 957, 912, 826.

19. The complex of any one of claims 1 to 18, wherein the complex is prepared as food, beverage, supplement or pharmaceutical preparation.

20. A 3-hydroxybutyric acid·3-hydroxybutyrate salt complex, which is obtained by the following method:
(1) a substance B is obtained by one of the following methods: mixing 3-hydroxybutyric acid and 3-hydroxybutyrate salt; or adding 3-hydroxybutyric acid into an aqueous solution of an alkaline compound, stirring, removing water, and evaporating to near dryness; or reacting alkyl 3-hydroxybutyrate with water under heating in the presence of a catalyst, cooling and filtering, adding an aqueous solution of an alkaline compound to the filtrate, distilling under reduced pressure to remove water, and evaporating to near dryness;
(2) adding one or more solvents selected from the following into the substance B obtained in step (1): water, THF, DMF, DMSO, DMAC, alcohol, halogenated hydrocarbon, ketone, ester, stirring and cooling, to precipitate a solid;
(3) filtering out the solid and drying, to obtain the complex.

21. The complex of claim 20, wherein the complex is a R-3-hydroxybutyric acid·R-3-hydroxybutyrate salt complex and/or a S-3-hydroxybutyric acid·S-3-hydroxybutyrate salt complex.

22. The complex of claim 20 or 21, wherein the complex is in crystalline form.

23. A method for preparing the complex of any one of claims 1 to 11, including the following steps:
(1) a substance B is obtained by one of the following methods: mixing 3-hydroxybutyric acid and 3-hydroxybutyrate salt; or adding 3-hydroxybutyric acid into an aqueous solution of an alkaline compound, stirring, removing water, and evaporating to near dryness; or reacting alkyl 3-hydroxybutyrate with water under heating in the presence of a catalyst, cooling and filtering, adding an aqueous solution of an alkaline compound to the filtrate, distilling under reduced pressure to remove water, and evaporating to near dryness;
(2) adding one or more solvents selected from the following into the substance B obtained in step (1): water, THF, DMF, DMSO, DMAC, alcohol, halogenated hydrocarbon, ketone, ester, stirring and cooling, to precipitate a solid;
(3) filtering out the solid and drying, to obtain the complex.

24. The method of claim 23, wherein in step (2), the alcohol is methanol, ethanol, isopropanol, n-butanol; the halogenated hydrocarbon is chlorobenzene, dichlorobenzene, dichloromethane; the ketone is acetone, methyl butanone, methyl isobutyl ketone; the ester is ethyl acetate, isopropyl acetate, n-butyl acetate, isobutyl acetate.

25. The method of claim 23 or 24, wherein in step (1), the alkaline compound is hydroxide, carbonate, bicarbonate, methanol, acetate or formate of potassium, calcium or magnesium; the alkyl 3-hydroxybutyrate is methyl 3-hydroxybutyrate, ethyl 3-hydroxybutyrate, propyl 3-hydroxybutyrate, isopropyl 3-hydroxybutyrate, butyl 3-hydroxybutyrate, isobutyl 3-hydroxybutyrate.

26. A composition comprising an effective amount of the complex of any one of claims 1 to 22, and a pharmaceutically acceptable carrier.

27. The composition of claim 26, wherein the composition is used as a ketogenic substance.

28. The composition of claim 26 or 27, wherein the composition is prepared as food, beverage, supplement or pharmaceutical preparation.

29. Use of the complex of any one of claims 1 to 22, the complex is used for preparing a ketogenic substance for increasing or maintaining blood ketone level of a subject.

30. The use of claim 29, wherein the ketogenic substance can be used as nutritional supplement, energy therapy, medical treatment or strength and/or endurance exercise supplement.

31. Use of a composition in preparing a ketogenic substance for increasing or maintaining blood ketone level of a subject, the composition comprises the complex of any one of claims 1 to 22, and a pharmaceutically acceptable carrier.

32. The use of claim 31, wherein the ketogenic substance can be used as nutritional supplement, energy therapy, medical treatment or strength and/or endurance exercise supplement.

33. A complex of acid and salt, wherein the acid comprises propionic acid, butyric acid, pentanoic acid, hexanoic acid and hydroxycarboxylic acid; and the salt comprises sodium salt, potassium salt, calcium salt and/or magnesium salt.

34. The complex of claim 33, wherein the hydroxycarboxylic acid is 3-hydroxypentanoic acid (BHP).

35. The complex of claim 33 or 34, wherein anions in the complex structure include 3-hydroxypentanoic acid anions; and cations include sodium ions and hydrogen ions.

36. The complex of claim 35, wherein the ratio of 3-hydroxypentanoic acid to sodium 3-hydroxypentanoate is 1:10 to 10:1.

37. The complex of any one of claims 33 to 36, wherein the complex contains not less than 50% of R configuration, not more than 50% of S configuration; or more than 50% of S configuration, less than 50% of R configuration.

38. The complex of any one of claims 33 to 37, wherein the complex has the following structure:

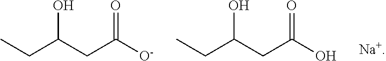

39. The complex of any one of claims 33 to 38, wherein the complex is in crystalline form.

40. The complex of any one of claims 33 to 39, wherein the complex is a R-3-hydroxypentanoic acid·sodium R-3-hydroxypentanoate and/or a S-3-hydroxypentanoic acid·sodium S-3-hydroxypentanoate.

41. The complex of any one of claims 33 to 40, wherein the complex contains not less than 50% of R-3-hydroxypentanoic acid·sodium R-3-hydroxypentanoate, not more than 50% of S-3-hydroxypentanoic acid·sodium S-3-hydroxypentanoate; or more than 50% of S-3-hydroxypentanoic acid·sodium S-3-hydroxypentanoate, less than 50% of R-3-hydroxypentanoic acid·sodium R-3-hydroxypentanoate.

42. The complex of claim 40 or 41, wherein the complex has the following structure:

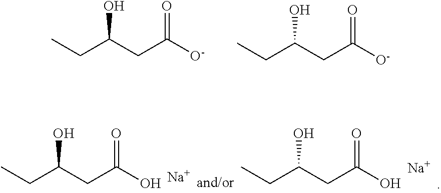

43. The complex of any one of claims 33 to 42, wherein the infrared spectrum of the complex has the following absorption bands, expressed in reciprocal wavelengths (cm⁻¹) (±2 cm⁻¹): 2968, 2880, 1715, 1558, 1404, 1065, 982, 912, 874, 783.

44. The complex of any one of claims 33 to 43, wherein the complex is prepared as food, beverage, supplement or pharmaceutical preparation.

45. A complex of acid and salt, which is obtained by the following method:
(1) a substance C is obtained by one of the following methods: mixing an acid and a corresponding salt; or adding an acid into an aqueous solution of an alkaline compound, stirring, removing water, and evaporating to near dryness; or reacting an alkyl ester of acid with water under heating in the presence of a catalyst, cooling and filtering, adding an aqueous solution of an alkaline compound to the filtrate, distilling under reduced pressure to remove water, and evaporating to near dryness;
(2) adding one or more solvents selected from the following into the substance C obtained in step (1): water, THF, DMF, DMSO, DMAC, alcohol, halogenated hydrocarbon, ketone, ester, stirring and cooling, to precipitate a solid;
(3) filtering out the solid and drying, to obtain the complex.

46. The complex of claim 45, wherein the complex is propionic acid·sodium propionate, propionic acid·potassium propionate, propionic acid·calcium propionate, propionic acid·magnesium propionate, butyric acid·sodium butyrate, butyric acid·potassium butyrate, butyric acid·calcium butyrate, butyric acid·magnesium butyrate, pentanoic acid·sodium pentanoate, pentanoic acid·potassium pentanoate, pentanoic acid·calcium pentanoate, pentanoic acid·magnesium pentanoate, hexanoic acid·sodium hexanoate, hexanoic acid·potassium hexanoate, hexanoic acid·calcium hexanoate, hexanoic acid·magnesium hexanoate, 3-hydroxypentanoic acid·sodium 3-hydroxypentanoate, 3-hydroxypentanoic acid·potassium 3-hydroxypentanoate, 3-hydroxypentanoic acid·calcium 3-hydroxypentanoate, 3-hydroxypentanoic acid·magnesium 3-hydroxypentanoate.

47. The complex of claim 45 or 46, wherein the complex is in crystalline form.

48. A method for preparing the complex of claim 33, including the following steps:
(1) a substance C is obtained by one of the following methods: mixing an acid and a corresponding salt; or adding an acid into an aqueous solution of an alkaline compound, stirring, removing water, and evaporating to near dryness; or reacting an alkyl ester of acid with water under heating in the presence of a catalyst, cooling and filtering, adding an aqueous solution of an alkaline compound to the filtrate, distilling under reduced pressure to remove water, and evaporating to near dryness;
(2) adding one or more solvents selected from the following into the substance C obtained in step (1): water, THF, DMF, DMSO, DMAC, alcohol, halogenated hydrocarbon, ketone, ester, stirring and cooling, to precipitate a solid;
(3) filtering out the solid and drying, to obtain the complex.

49. The method of claim 48, wherein in step (2), the alcohol is methanol, ethanol, isopropanol, n-butanol; the halogenated hydrocarbon is chlorobenzene, dichlorobenzene, dichloromethane; the ketone is acetone, methyl butanone, methyl isobutyl ketone; the ester is ethyl acetate, isopropyl acetate, n-butyl acetate, isobutyl acetate.

50. The method of claim 48 or 49, wherein in step (1), the alkaline compound is hydroxide, carbonate, bicarbonate, methanol, acetate or formate of sodium, potassium, calcium or magnesium; the alkyl ester of acid is methyl ester, ethyl ester, propyl ester, isopropyl ester, butyl ester and isobutyl ester of acid.

51. A composition comprising an effective amount of the complex of any one of claims 33 to 47, and a pharmaceutically acceptable carrier.

52. The complex of claim 51, wherein the composition is used as a ketogenic substance.

53. The complex of claim 51 or 52, wherein the composition is prepared as food, beverage, supplement or pharmaceutical preparation.

54. Use of the complex of any one of claims 33 to 47, the complex is used for preparing a ketogenic substance for increasing or maintaining blood ketone level of a subject.

55. The use of claim 54, wherein the ketogenic substance can be used as nutritional supplement, energy therapy, medical treatment or strength and/or endurance exercise supplement.

56. Use of a composition in preparing a ketogenic substance for increasing or maintaining blood ketone level of a subject, the composition comprises the complex of any one of claims 33 to 47, and a pharmaceutically acceptable carrier.

57. The use of claim 56, wherein the ketogenic substance can be used as nutritional supplement, energy therapy, medical treatment or strength and/or endurance exercise supplement.
